(19) United States
(12) Patent Application Publication
(10) Pub. No.: US 2015/0211053 A1
(43) Pub. Date: Jul. 30, 2015

(54) BIOMARKERS FOR DIABETES AND USAGES THEREOF

(71) Applicants: Shenghui Li, Shenzhen (CN); Qiang Feng, Shenzhen (CN); Junjie Qin, Shenzhen (CN); Jianfeng Zhu, Shenzhen (CN); Dongya Zhang, Shenzhen (CN); Zhuye Jie, Shenzhen (CN); Jun Wang, Shenzhen (CN); Jian Wang, Shenzhen (CN); Huanming Yang, Shenzhen (CN)

(72) Inventors: Shenghui Li, Shenzhen (CN); Qiang Feng, Shenzhen (CN); Junjie Qin, Shenzhen (CN); Jianfeng Zhu, Shenzhen (CN); Dongya Zhang, Shenzhen (CN); Zhuye Jie, Shenzhen (CN); Jun Wang, Shenzhen (CN); Jian Wang, Shenzhen (CN); Huanming Yang, Shenzhen (CN)

(73) Assignee: BGI-Shenzhen, Shenzhen (CN)

(21) Appl. No.: 13/639,781

(22) PCT Filed: Sep. 3, 2012
(86) PCT No.: PCT/CN2012/080922

§ 371 Date: Apr. 13, 2015

(30) Foreign Application Priority Data
Aug. 1, 2012 (CN) PCT/CN2012/079522

(51) Int. Cl. C12Q 1/68

(52) U.S. Cl. CPC C12Q 1/689; C12Q 2600/118; C12Q 2600/16

(57) ABSTRACT

Biomarkers for diabetes and usages thereof are provided. And the biomarkers are Akkermansia muciniphila, Bacteroides intestinalis, Bacteroides sp. 20_3, Clostridium bolteae, Clostridium hathewayi, Clostridium ramosum, Clostridium sp. HGF2, Clostridium symbiosum, Desulfovibrio sp. 3_1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans.

### CROSS-REFERENCE TO RELATED APPLICATION

The present patent application claims benefit of priority to PCT Patent Application No. PCT/CN2012/079522, filed Aug. 1, 2012, which is incorporated herein by reference.

### TECHNOLOGY FIELD

The present invention relates to the field of biomedicine, specifically related to diabetes markers and its applications.

### BACKGROUND

Diabetes has become the third serious threat to human health of chronic diseases for the world, following after cancer, cardiovascular and cerebrovascular disease. At the same time, it will seriously affect the heart and brain blood vessels and kidneys. With the rapid economic development and way of life continuing to improve, the incidence rate of diabetes and other metabolic diseases sharp rises, which has become a major threat to human health. The latest statistic shows that, according to the International Diabetes Federation, the incidence of diabetes reached 2.5% in 1994, while 5.5% in 2002 and 9.7% in 2008. At present, the incidence of diabetes in China makes no difference with that of economically developed America, the big cities have reached 9-10%. In 2005, the World Health Organization released a report that from 2005 to 2015, heart disease, stroke and diabetes would lead to premature death and a loss of about 3.9 trillion RMB in national income. Therefore, the research of major cause of diabetes, and the establishment of a powerful and easy to promote interventions to curb the rising trend of the incidence of diabetes in the population, has become China's scientific problems in the field of biomedicine and nutrition.

More than 90% of population with diabetes are type II diabetes. Type II diabetes is a chronic integrated disease due to blood glucose self-imbalance, performing the symptoms of high blood sugar. During the progress of the disease, it causes disorders of carbohydrate and fat metabolism, affecting normal physiological activity of body organs organization. Pathological causes of Type II diabetes are more diversified, generally considered to be innate genetic factors and acquired environmental factors together. For the study of these areas, there are many, but they can not explain well the occurrence of type II diabetes and the pathogenesis.

At present, the research of type II diabetes still needs to be improved.

### SUMMARY

The present invention is based on the following findings of the inventor: Innate genetic factors can only explain less than 5% of patients with diabetes. Current study neglects an important issue, which is the intestinal microflora. The intestinal microbes called “the second genome” grow in the human intestinal microbial community. Human intestinal flora and the host constitutes an interrelated whole. Gut microbes are not only capable of degrading to digest nutrients in food, host vitamins and other nutrients, but also promoting the differentiation and maturation of the intestinal epithelial cells to activate the intestinal immune system and the regulation of host energy storage and metabolism, which have played an important role in digestion and absorption, immune response, metabolic activity in the body. Intestinal flora can also control fat metabolism in animals and low-grade chronic inflammation caused by systemic, leading to obesity and insulin resistance, and this pathogenic role is far greater than the contribution of animal genetic defects. The applicant filtered out the high correlation of biomarkers with type II diabetes through the intestinal flora, and used the markers to diagnose type II diabetes correctly, and monitor treatment effect.

According to one embodiment of present disclosure, a group of isolated microbes is provided wherein the group consisting of Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans. Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans are T2D biomarkers. By determining presence or absence of at least one of these microbes in gut microbiota, one may effectively determine whether a subject has or is susceptible to T2D, and monitor treatment effect of patients with T2D. Through determining relative abundances of at least one of these microbes and comparing the abundances with predicted critical values, one may promote the efficiency of determining whether a subject has or is susceptible to T2D, and monitoring treatment effect of patients with T2D.

According to one embodiment of present disclosure, a method to determine abnormal condition in a subject is provided comprising the step of determining presence or absence of Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans in gut microbiota. Using this method, one may determine relative abundances of these microbes in gut microbiota and then compare the obtained relative abundances with predicted critical values (Cut off) so as to promote the efficiency of determining whether a subject has or is susceptible to T2D, and monitoring treatment effect of patients with T2D.

According to one embodiment of present disclosure, a system to determine abnormal condition in a subject is provided comprising: nucleic acid sample isolation apparatus, which adapted to isolate nucleic acid sample from the subject; sequencing apparatus, which connected to the nucleic acid sample isolation apparatus and adapted to sequence the nucleic acid sample, to obtain a sequencing result; and alignment apparatus, which connect to the sequencing apparatus, and adapted to align the sequencing result against the reference genomes in such a way that determine the presence or absence of Akkermansia muciniphila, Bacteroides intestinalis, Bacteroides sp. 20_{—}3, Clostridium bolteae, Clostridium hathewayi, Clostridium ramosum, Clostridium sp. HGF2. Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans. Using this method, one may determine relative abundances of these microbes in gut microbiota and then compare the obtained relative abundances with predicted critical values (Cut oft) so as to promote the efficiency of determining whether a subject has or is susceptible to T2D, and monitoring treatment effect of patients with T2D.

According to one embodiment of present disclosure, a kit for determining abnormal condition in a subject, is provided which is adapted to determine Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans. By means the above kit, one may determine relative abundances of these microbes in gut microbiota and then compare the obtained relative abundances with predicted critical values (Cut off) so as to promote the efficiency of determining whether a subject has or is susceptible to T2D, and monitoring treatment effect of patients with T2D.

According to one embodiment of present disclosure, the usage of biomarkers as target for screening medicaments to treat or prevent abnormal conditions is provided, in which the biomarkers are Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, and the abnormal condition is diabetes, optionally, Type 2 Diabetes. One may use the effect on these microbes before and after drug candidate administration to determine whether the drug candidate can be used as T2D drugs for treatment or prevention.

Additional aspects and advantages of embodiments of present disclosure will be given in part in the following descriptions, become apparent in part from the following descriptions, or be learned from the practice of the embodiments of the present disclosure.

### BRIEF DESCRIPTION OF DRAWINGS

These and other aspects and advantages of the present disclosure will become apparent and more readily appreciated from the following descriptions taken in conjunction with the drawings, in which:

FIG. 1 shows the flow diagram of the system to determine abnormal condition in a subject according to one embodiment of present disclosure.

FIG. 2 to 4 show the flow diagram of the method to determine biomarkers related to Type 2 Diabetes according to embodiment 3, 4, and 5 of present disclosure.

FIG. 5, according to one embodiment of present disclosure, shows detection error rate distribution of relative abundance profiles in different sequencing amount. The X axis represents the sequencing amount of a sample, which was defined as the number of paired-end reads, and the Y axis represents the relative abundance of a gene. The 99% confidence interval (CI) of the relative abundance was estimated and the detection error rate was defined as the ratio of the interval width to the relative abundance itself. The scaled detection error rate, transformed by log₁₀(log₁₀(1+x)), was used to color all the points, with warmer color representing larger detection error rate. Two indifference curves were added: detection error rate that fall to the upper right of the curves would be less than 1× and 10×, respectively.

FIG. 6 (A1-A6): In the growth curves, during the 8 weeks after introduction of high-fat diet, body weight increased significantly more in the high-fat diet-fed mice, which 10.4±1.4 g than in the normal diet-fed mice (4.5±0.1 g; P<0.001). And the body weight of HF fed with 11 strains of bacteria (group B1-B6) was significantly lower than HF group (P<0.05), which suggested that the fermentation liquid could help with the mitigation of obesity development. FIG. 6 (A7-17): Effects of strains administration on body weight in normal mice fed a high fat die or chow diet. A7-A17: The mice treated with B7-B17 demonstrated increases in body weight (group B7-B17) comparing with high-fat diet-fed mice (group A) during the 8 weeks, and most of the increases were significant.

### DETAILED DESCRIPTION

In the following detailed description of the embodiments of present disclosure, the embodiment examples are shown in the drawings, wherein the same or a similar label to the same or similar elements or components of the same or similar functions. The following embodiments described by reference drawings are exemplary, which only used to explain the present invention, and not regarded as the limitations of the present invention.

### Biomarkers

According to embodiments of a first broad aspect of the present disclosure, biomarkers related to Type 2 Diabetes are provided.

According to the embodiment of present disclosure, the term “biomarker” should have a broad understanding, that is any detectable biological indicators reflecting the abnormal condition, which comprises gene marker, species marker (species/genus marker) and functions marker (KO/OG marker). The meaning of gene markers is not only existing expression of the gene for biologically active proteins, but also includes any nucleic acid fragment: DNA, RNA, modified and unmodified. The gene markers can sometimes also be called the characteristic fragments.

According to the embodiment of present disclosure, the high-throughput sequencing is used to analysis health and T2D feces samples in batch. Based on high-throughput sequencing data, conduct statistical tests on the health and T2D group, and then determine specific nucleotide sequences related to T2D group In short, the following steps comprise:

samples collection and storage, wherein the feces samples are collected from health and T2D group, and then DNA extraction is conducted by using kits to obtain nucleic acid samples.

library construction and sequencing, wherein DNA library construction and sequencing are performed by high-throughput sequencing in order to obtain nucleotide sequences of gut microbiota in the feces samples.

Determine specific nucleotide sequences of gut microbiota related to T2D group based on bioinformation analysis. First, align the sequencing results (reads) against reference gene catalogue (gene catalogue newly constructed or any known database, for example the human gut microbial non-redundant gene catalogue). Next, determine relative abundance of gene respectively in the nucleic acid samples from the health and T2D group based on the alignment result; By aligning the sequencing reads against reference gene catalogue, built up a corresponding relationship between sequencing reads and gene of reference gene catalogue. So that corresponding sequence reads relative number can reflect the gene relative abundance effectively, aiming at specific gene of nucleic acid samples. Thus, through alignment result and conventional statistic analysis, determine the relative gene abundance in the nucleic acid samples. Finally, after determining relative abundance of gene in the nucleic acid samples, conduct statistical tests on the relative abundance of gene in the nucleic acid samples from the health and T2D group in order to determine gene markers which are significantly different between the nucleic acid samples from the health and T2D group based on their relative abundances. If the existing gene is significantly different, the gene is regarded as biomarker related to abnormal condition, namely gene marker.

In addition, as for the known or newly constructed reference gene catalogue, the taxonomic assignment and functional annotation of gene may be included. In this way, based on the gene relative abundances, perform taxonomic assignment and functional annotation of gene, and then determine species and functions relative abundances of the gut microbiota. Further, determine species and functions markers related to abnormal condition. In short, determining the species and functions markers further comprises: aligning sequencing results against reference gene catalogue; and determining species and functions relative abundances of gene respectively in the nucleic acid samples from the health and T2D group based on the alignment result; and conducting statistical tests on the species and functions relative abundances of gene in the nucleic acid samples from the health and T2D group; and determining species and functions markers respectively which are significantly different between the nucleic acid samples from the health and T2D group based on their relative abundances. According to the embodiment of present disclosure, conduct statistical tests on the gene relative abundances from the same species and from the same functional annotation respectively, for example summation, average, median values and so on, to determine species and functions relative abundances.

Finally, microbes which are significantly different between the feces samples from the health and T2D group based on their relative abundances are determined, namely Akkermansia muciniphilae, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans. One may determine the presence or absence of at least one of these microbes to determine whether a subject has or is susceptible to T2D, and monitor treatment effect of patients with diabetes. Used herein, the term “presence” should have a broad understanding of the qualitative analysis of samples on that whether the sample contains the corresponding target, or the quantitative analysis of the target in the sample. Furthermore, one may also conduct statistical analysis or any known mathematical algorithm on obtained quantitative results and reference results (for example, quantitative results from parallel testing of samples with known condition). Skilled in the art can base on the needs and test conditions to choose easily. One may determine relative abundances of these microbes in gut microbiota and then compare the obtained relative abundances with predicted critical values (Cut off) so as to promote the efficiency of determining whether a subject has or is susceptible to T2D, and monitoring treatment effect of patients with T2D.

According to the embodiment of present disclosure, the microbes, Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli, which are enriched in T2D group, are called harmful biomarkers. Clostridiales sp. SS3/4, Eubacterium rectale, Faecalibacterium prausnitzii, Haemophilus parainfluenzae, Roseburia intestinalis and Roseburia inulinivorans, which are enriched in healthy group (control group), and are called beneficial biomarkers.

One may determine the presence or absence of at least one of these microbes, especially Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli, to determine whether a subject has or is susceptible to T2D, and monitor treatment effect of patients with diabetes.

### A Method to Determine Abnormal Condition in a Subject

According to one embodiments of present disclosure, a method to determine abnormal condition in a subject is provided comprising the step of determining presence or absence of nucleotides having at least one of polynucleotide sequences defined in Table 9 in a gut microbiota of the subject, namely at least one of gene markers, species markers and functions markers which mentioned above.

According to one embodiment of present disclosure, the abnormal condition is diabetes, preferably, Type 2 Diabetes. One may determine the presence or absence of at least one of the biomarkers above to determine whether a subject has or is susceptible to T2D, and monitor treatment effect of patients with diabetes.

According to one embodiment of present disclosure, determining the presence or absence of at least one of these microbes in gut microbita, Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, especially Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_syn3, Eggerthella lenta and Escherichia coli, further comprises: DNA extraction from excreta, library construction and sequencing. One may obtain sequencing results and then determine the presence or absence of at least one of these microbes in excreta. Through sequencing, one may obtain the subjects' nucleic acid data in gut microbiota and then effectively determine the presence or absence of gene markers.

According to the embodiment of present disclosure, the sequencing technologies are not limited. The sequencing step is conducted by means of second-generation sequencing method or third-generation sequencing method, preferably by means of at least one apparatus selected from Hiseq 2000, SOLID, 454, and True Single Molecule Sequencing. In this way, one can take advantage of features of high throughput and depth sequencing from the sequencing apparatus, which benefits the following data analysis, especially statistical test in precision and accuracy.

One may align the sequencing result against the reference genomes in such a way that determine the presence or absence of the microbes mentioned above, for example that the reference genomes comprise the known genomes information of detected microbes. The step of aligning is conducted by means of at least one of SOAP 2 and MAQ. In this way, it helps to improve efficiency of alignment and then improve efficiency of determining abnormal condition, optionally, T2D. Meanwhile, more (at least two) biomarkers can be determined so as to improve efficiency of determining abnormal condition, optionally, T2D.

For species markers and functions markers, skilled in the art can determine the presence or absence of the species and functions in gut microbiota by conventional microbe identification method and biological activity test. For example, microbe identification can be conducted by 16s rRNA method.

According to one embodiment of present disclosure, the method further comprises the steps of: determining relative abundances of at least one of Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, especially Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli; and comparing the abundances with predicted critical values. Based on the difference between the abundances and predicted critical values, one may determine whether a subject has abnormal condition. The predicted critical values can be obtained by conventional experiment, for example by determining relative abundances of biomarkers in the subject through parallel testing of samples with known physiological status. The predicted critical values (cutoff) are shown in the table below. For beneficial species maker (direction defined as 0), if the test sample's relative abundance is less than the best cutoff then the inventors predict the test sample is in disease condition. For harmful species maker (direction defined as 1), if the test sample's relative abundance is larger than the best cutoff then the inventors predict the test sample is in disease condition.

| type | microbes | cutoff |
|---|---|---|
| harmful species makers | Clostridium bolteae | 0.103658 |
| | Escherichia coli | 0.498151 |
| | Bacteroides sp. 20_3 | 1.553228 |
| | Bacteroides intestinalis | 0.49045 |
| | Akkermansia muciniphila | 8.95E−05 |
| | Clostridium symbiosum | 0.00508 |
| | Desulfovibrio sp. 3_1_syn3 | 0.098314 |
| | Clostridium sp. HGF2 | 0.015788 |
| | Clostridium hathewayi | 0.000673 |
| | Eggerthella lenta | 0.046154 |
| | Clostridium ramosum | 0.003178 |
| beneficial species | Clostridiales sp. SS3/4 | 0.34953 |
| makers | Eubacterium rectale | 0.059392 |
| | Roseburia inulinivorans | 0.36604 |
| | Roseburia intestinalis | 0.06585 |
| | Faecalibacterium prausnitzii | 0.663083 |
| | Haemophilus parainfluenzae | 0.001912 |

Clostridiales sp. SS3/4, Eubacterium rectale, Faecalibacterium prausnitzii, Haemophilus parainfluenzae, Roseburia intestinalis and Roseburia inulinivorans can be used as beneficial bacteria to treat or prevent T2D. For example, these beneficial bacteria can be used in food. According to one embodiment of present disclosure, a food or pharmaceutical composition is provided, wherein the food or pharmaceutical composition comprises at least one of Clostridiales sp. SS3/4, Eubacterium rectale, Faecalibacterium prausnitzii, Haemophilus parainfluenzae, Roseburia intestinalis and Roseburia inulinivorans. Using this food or pharmaceutical composition can prevent or treat T2D effectively. In addition, a usage is provided of at least one of Clostridiales sp. SS3/4, Eubacterium rectale, Faecalibacterium prausnitzii, Haemophilus parainfluenzae, Roseburia intestinalis and Roseburia inulinivorans in the preparation of composition for prevention and/or treatment of T2D. Also a method to treat T2D is provided, comprising administrating Clostridiales sp. SS3/4, Eubacterium rectale, Faecalibacterium prausnitzii, Haemophilus parainfluenzae, Roseburia intestinalis and Roseburia inulinivorans to the subjects in need.

### A System to Determine Abnormal Condition in a Subject

According to one embodiment of present disclosure, a system (1000) is provided to determine abnormal condition in a subject. The system comprises nucleic acid sample of gut microbiota isolation apparatus and biomarkers determination apparatus. For different types of biomarkers, one may use related nucleic acid sample of gut microbiota isolation apparatus and biomarkers determination apparatus.

For gene markers, referring to FIG. 1, the system to determine abnormal condition in a subject comprises: nucleic acid sample isolation apparatus (100), sequencing apparatus (200) and alignment apparatus (300). Nucleic acid sample isolation apparatus which adapted to isolate nucleic acid sample of gut microbiota from the subject. Sequencing apparatus (200) is connected to the nucleic acid sample isolation apparatus (100) and adapted to sequence the nucleic acid sample to obtain a sequencing result. Alignment apparatus (300) is connected to the sequencing apparatus (200) and adapted to align the sequencing result against reference genomes in such a way that determine the presence or absence of at least one of Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, especially Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli. The reference genomes comprise at least one of microbial genomes of Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, especially Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli. By means the above system, one may conduct any previous method to determine abnormal condition so as to effectively determine the presence or absence of at least one of Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, especially Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli, and then one may determine whether there is abnormal condition in the subject effectively.

According to one embodiment of present disclosure, the abnormal condition is diabetes, preferably Type 2 Diabetes. At least one of Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, especially Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli, are T2D biomarkers. One may determine the presence or absence of at least one of these biomarkers to determine whether a subject has or is susceptible to T2D, and monitor treatment effect of patients with T2D. The nucleic acid sample isolation apparatus is adapted to isolate nucleic acid sample of gut microbiota from faces.

According to the embodiment of present disclosure, the sequencing technologies are not limited. Preferably, the sequencing step is conducted by means of next-generation sequencing method or next-next-generation sequencing method, preferably by means of at least one apparatus selected from Hiseq 2000, SOLID, 454, and True Single Molecule Sequencing. In this way, one can take advantage of features of high throughput and depth sequencing from the sequencing apparatus, which benefits the following data analysis, especially statistical test in precision and accuracy.

According to one embodiment of present disclosure, the alignment apparatus is at least one of SOAP 2 and MAQ. In this way, it helps to improve efficiency of alignment and then improve efficiency of determining abnormal condition, optionally T2D.

For species markers and functions markers, skilled in the art can determine the presence or absence of the species and functions in gut microbiota by conventional microbe identification method and biological activity test. For example, microbe identification can be conducted by 16s rRNA method.

### Others

According to one embodiment of present disclosure, a kit for determining abnormal condition in a subject is provided, including the reagents which adapted to determine at least one of the biomarkers above. For gene markers, the kit comprises reagents adapted to determine at least one of Akkermansia muciniphila, Bacteroides intestinalis. Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1syn3, Eggerthella lenta, Escherichia coil Clostridiales sp. SS3/4, Eubacterium rectale, Faecalibacterium prausnitzii, Haemophilus parainfluenzae, Roseburia intestinalis and Roseburia inulinivorans, especially Akkermansia muciniphilae, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli. By the system, one may determine the presence or absence of at least one of Akkermansia muciniphilae, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, especially Akkermansia muciniphilae, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli effectively, and then one may determine whether there is abnormal condition in the subject. The abnormal condition is diabetes, preferably Type 2 Diabetes.

In addition, according to embodiments of the present disclosure, a method of screening medicaments is provided. Using T2D biomarkers as target to screen medicaments can promote new T2D drugs discovery. For example, one can detect the changes of the biomarkers' level before and after drug candidates' administration to determine whether the drug candidate can be used as T2D drugs for treatment or prevention. For example that one can determine whether the harmful markers' level decrease and whether the beneficial markers' level increase after drug candidates' administration. Specially, one may also determine the drugs' direct or indirect effect on at least one of Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans, especially Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta and Escherichia coli to determine whether the drug candidate can be used as T2D drugs for treatment or prevention. According to embodiments of the present disclosure, there is provided a usage of T2D biomarkers as target for screening medicaments to treat or prevent T2D.

The present invention is further exemplified in the following non-limiting examples.

Unless otherwise stated, the technical means used in the examples are well-known conventional to the skilled in the art, referring to “Laboratory Manual For Molecular Cloning” (third edition) or related products, and the reagents and products are all commercially available. Not stated in detail, the various processes and methods are conventional to the public in this field, and the source of the reagents, trade names and its composition needed to set out are indicated when it first appears. Unless otherwise stated, the same reagents used subsequently are in accordance with the first indicated instructions.

### Example 1

### Sample Collection

All 344 faecal samples from 344 Chinese individuals living in the south of China were collected by Shenzhen Hospital of Peking University. The patients who were diagnosed with type 2 diabetes (T2D) Mellitus according to the 1999 WHO criteria (Alberti, K. G & Zimmet, P. Z. Definition, diagnosis and classification of diabetes mellitus and its complications. Part 1: diagnosis and classification of diabetes mellitus provisional report of a WHO consultation. Diabetic medicine: a journal of the British Diabetic Association 15, 539-553,doi: 10.1002/(SICI) 1096-9136 (199807) 15:7<539::AID-DIA668>3.0.CO;2-S (1998), incorporated herein by reference) constitute the case group in the study, and the rest non-diabetic individuals were taken as the control group (shown in Table I). Patients and healthy controls were asked to provide a frozen faecal sample. Volunteers pay attention to 3 days' diet before sampling, and eat light, but not high fat foods. And in the 5 days before sampling, volunteers didn't eat yogurt and other lactic acid products and prebiotics. The samples were collected not to mix with urine, and isolated from human pollution and air.

TABLE 1
Sample collection

Samples

| Sample | T2D | Obesity | Stage I | Stage II |
|---|---|---|---|---|
| DO | Yes | Yes | 32 | 73 |
| DL | Yes | No | 39 | 26 |
| NO | No | Yes | 37 | 62 |
| NL | No | No | 37 | 38 |

### Example 2

### DNA Extraction and Sequencing

2.1 Faecal Samples Storage

Fresh faecal samples were taken into the sterilized stool collection tube, and samples were immediately frozen by storing in a home freezer. Frozen samples were transferred to the place to store, and then stored at −80° C. until analysis.

2.2 DNA Extraction

A frozen aliquot (200 mg) of each fecal sample was suspended in 250 μl of guanidine thiocyanate, 0.1 M Tris (pH 7.5) and 40 μl of 10% N-lauroyl sarcosine. DNA was extracted as previously described (Manichanh, C. et al. Reduced diversity of faecal microbiota in Crohn's disease revealed by a metagenomic approach. Gut 55, 205-211, doi:gut. 2005.073817 [pii]0.1136/gut.2005.073817 (2006), incorporated herein by reference). DNA concentration and molecular size were estimated using a nanodrop instrument (Thermo Scientific) and agarose gel electrophoresis.

2.3 DNA Library Construction and Sequencing

DNA library construction was performed following the manufacturer's instruction (Illumina). The inventors used the same workflow as described elsewhere to perform cluster generation, template hybridization, isothermal amplification, linearization, blocking and denaturation, and hybridization of the sequencing primers.

The inventors constructed one paired-end (PE) library with insert size of 350 bp for each samples, followed by a high-throughput sequencing to obtain around 20 million PE reads. The reads length for each end is 75 bp-90 bp (75 bp and 90 bp read length in stage I samples; 90 bp read length for stage 11 samples).

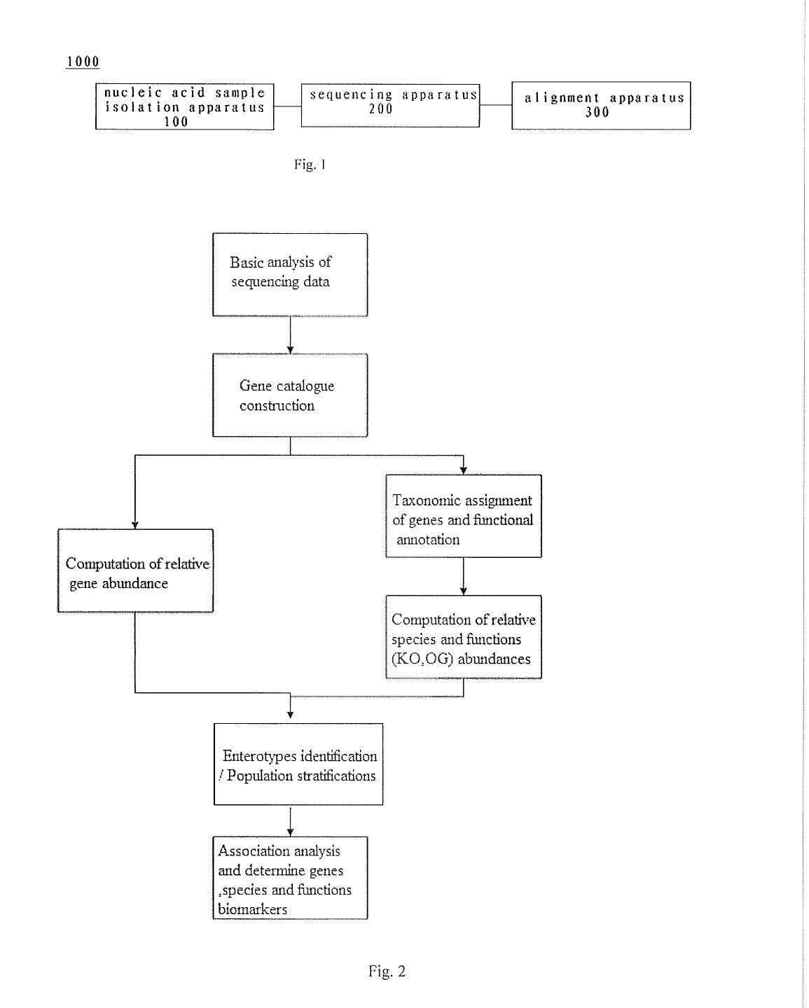

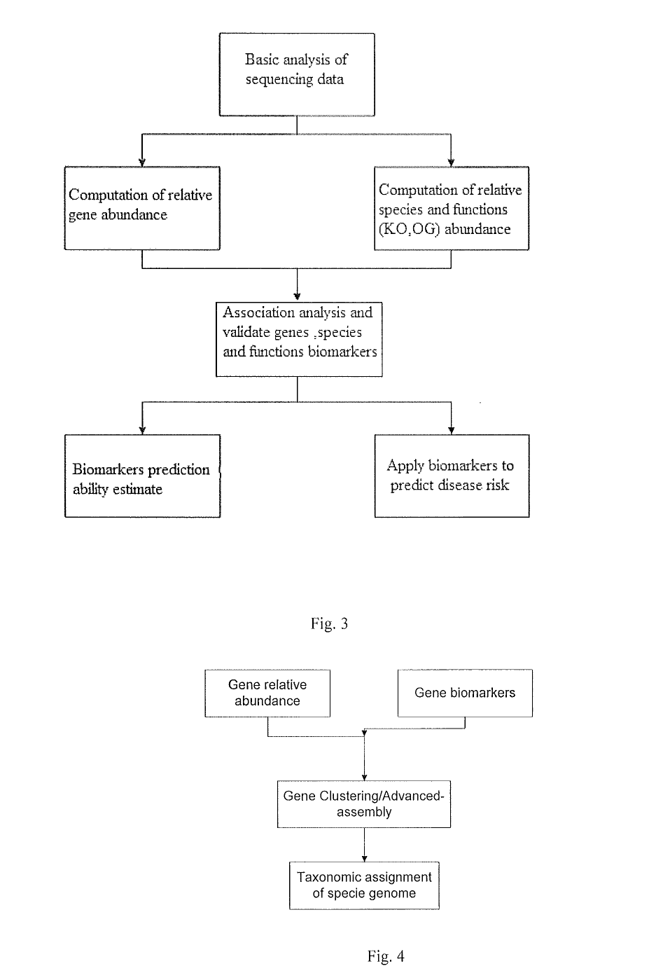

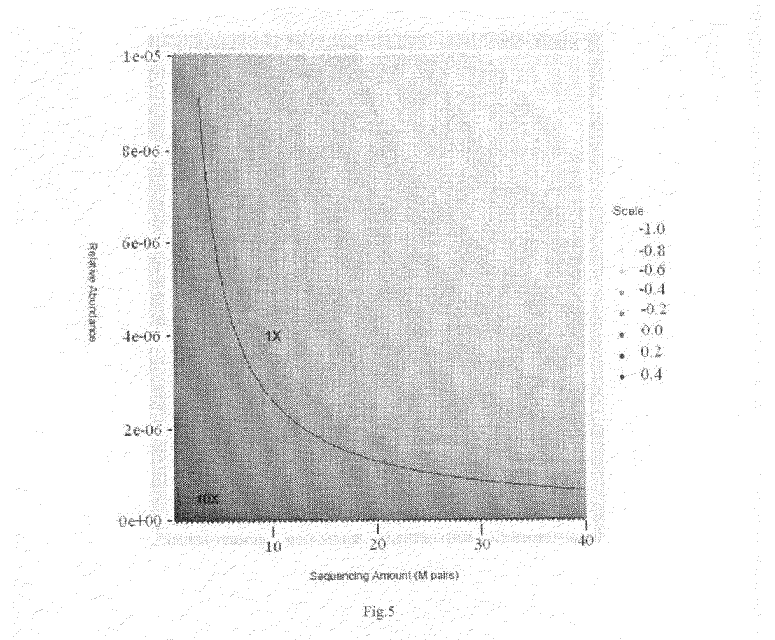

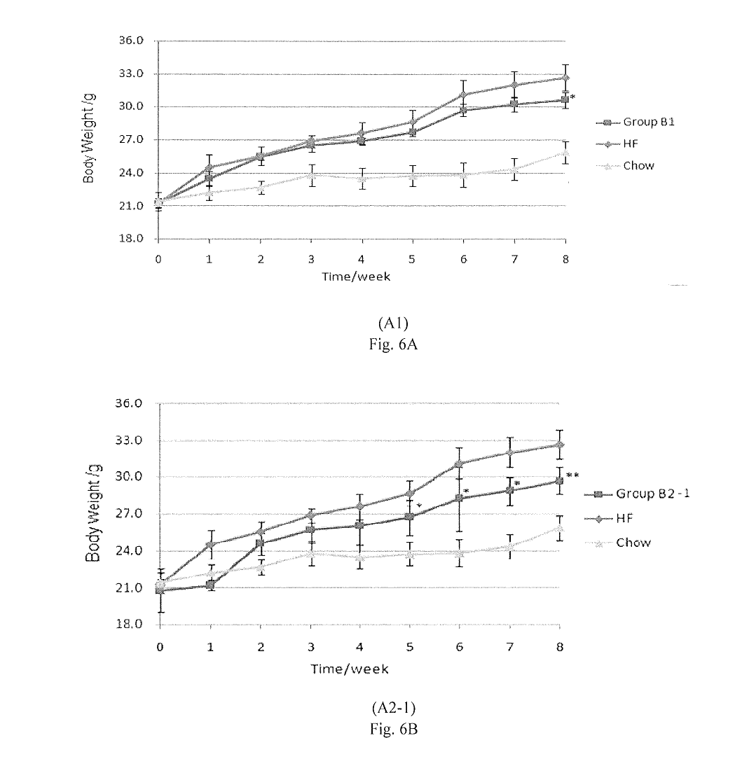

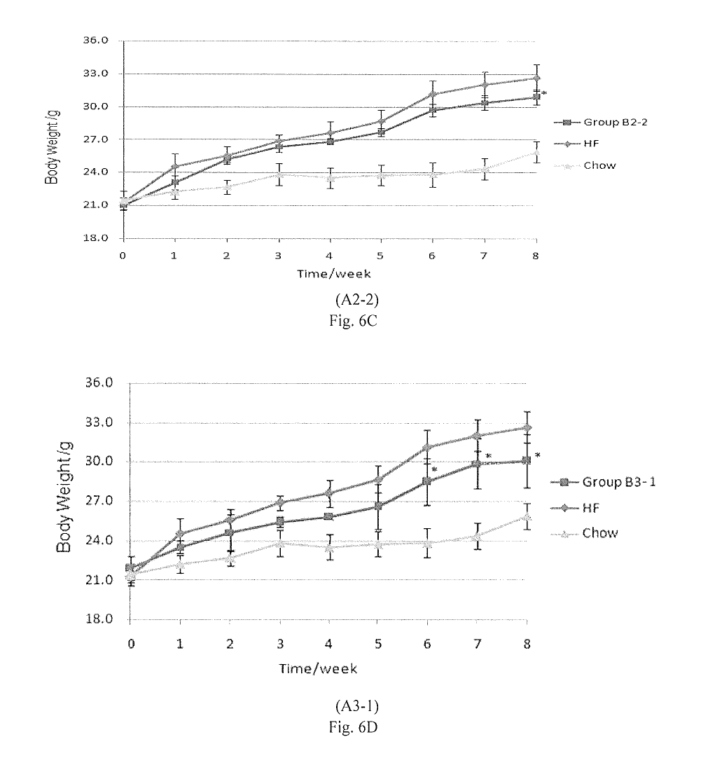

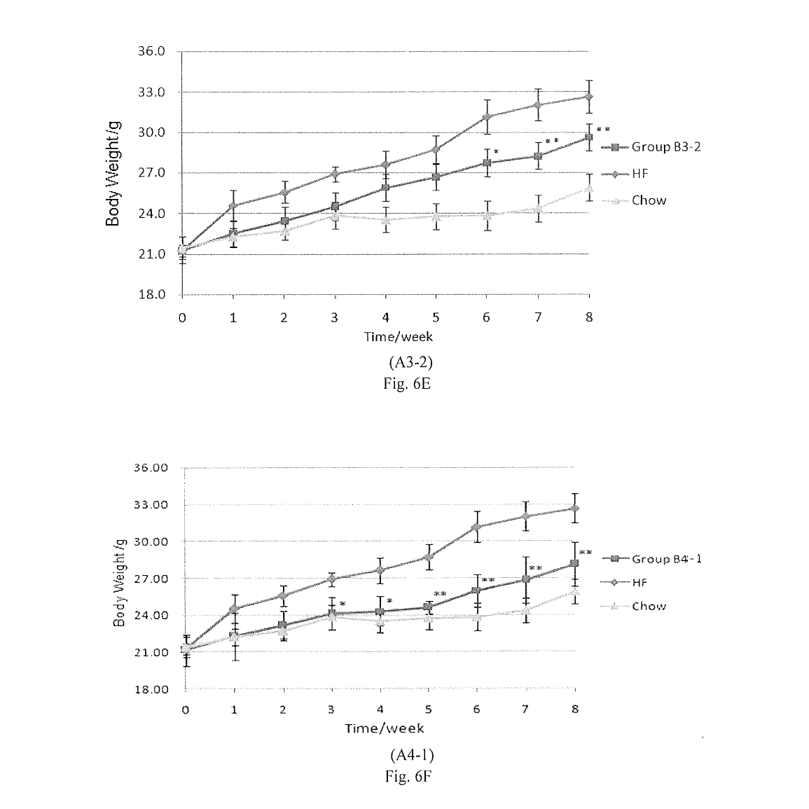

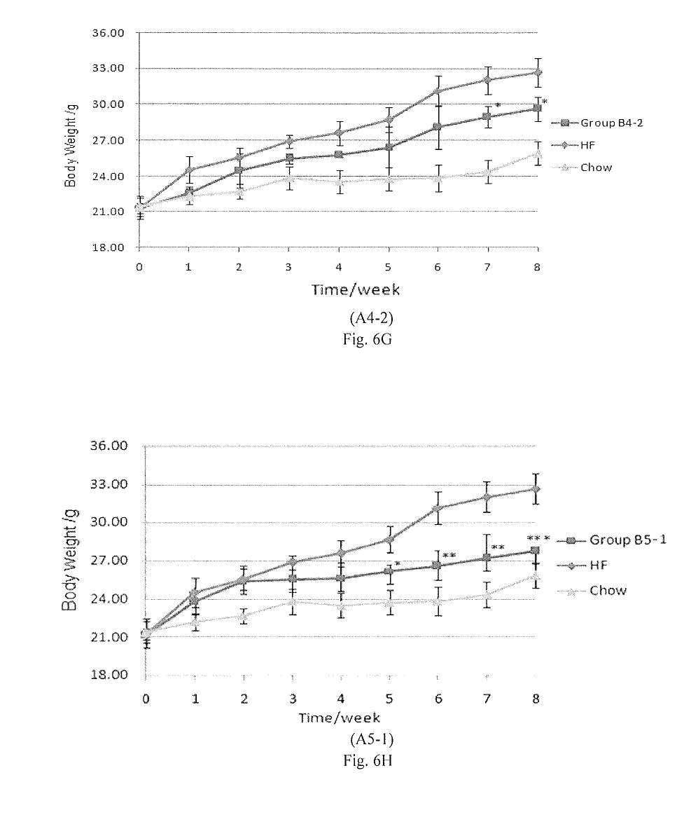

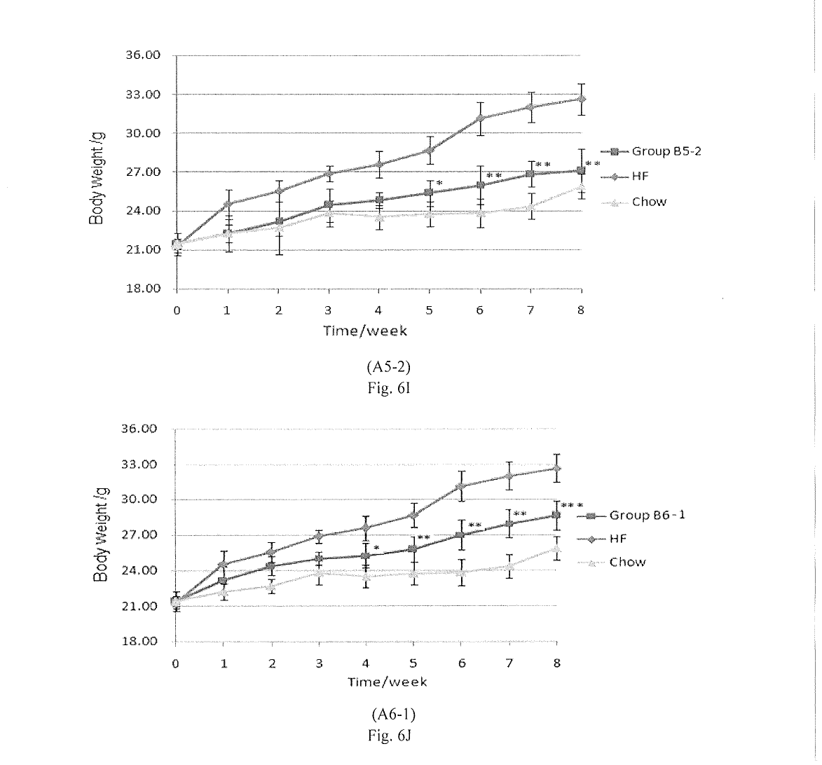

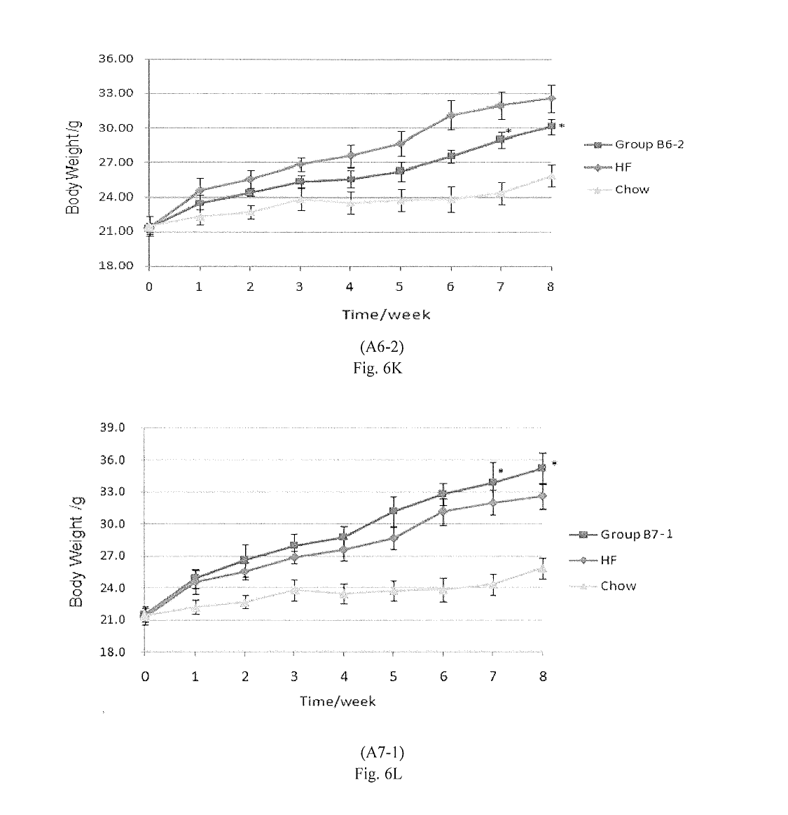

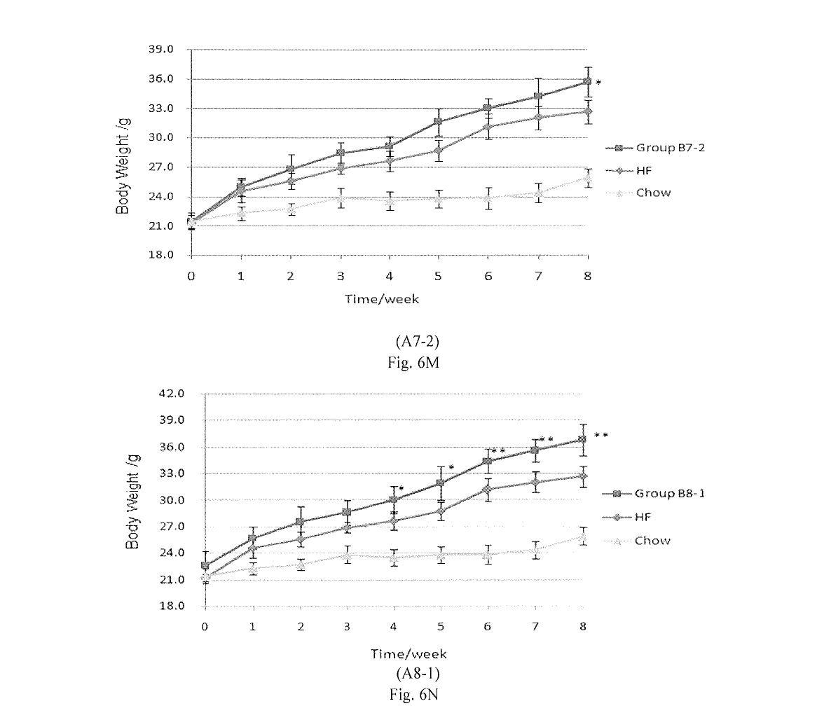

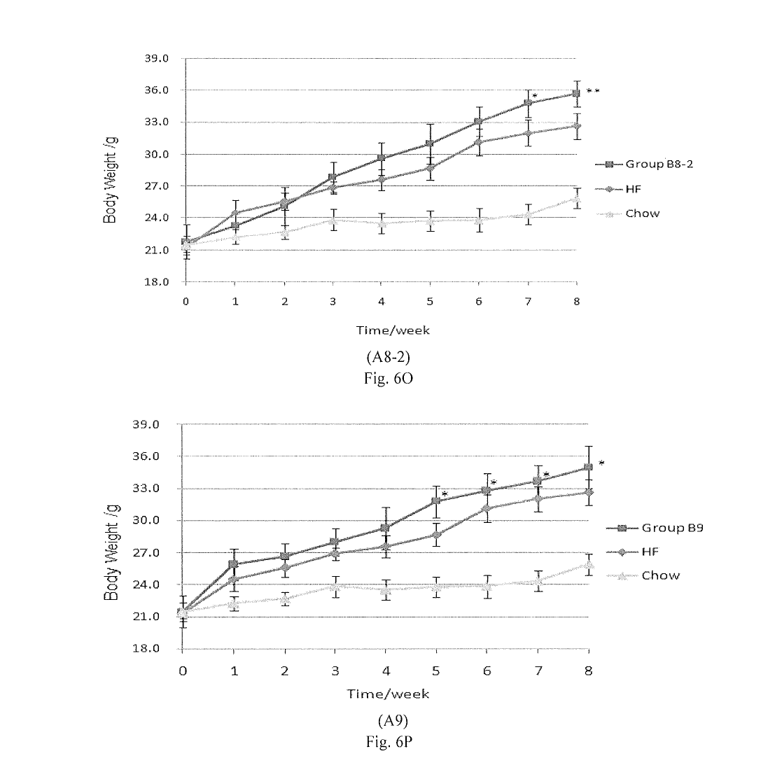

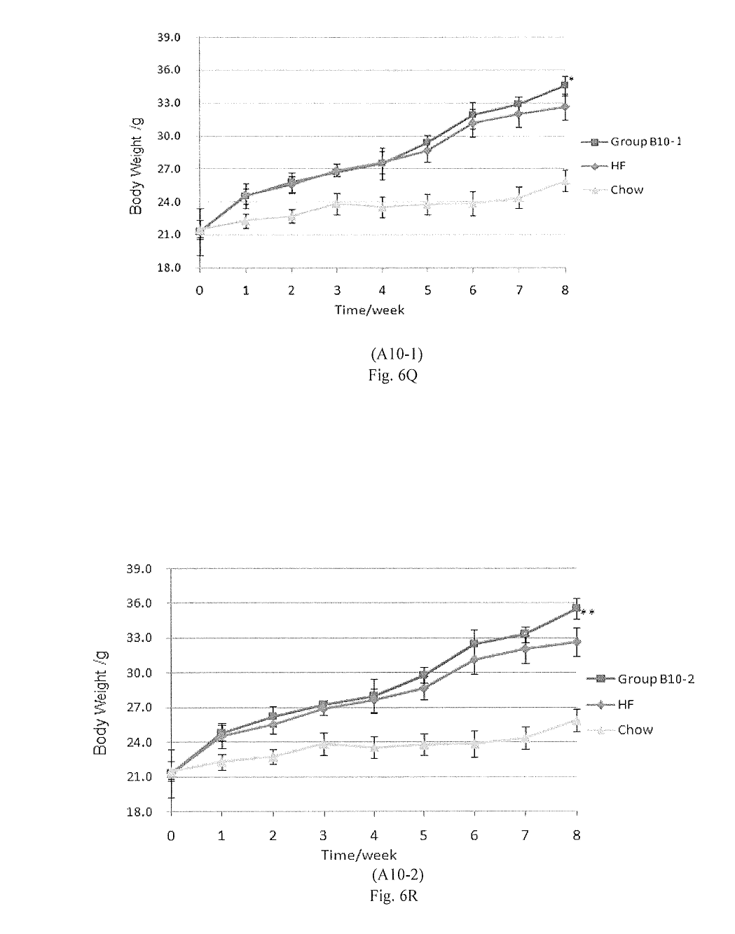

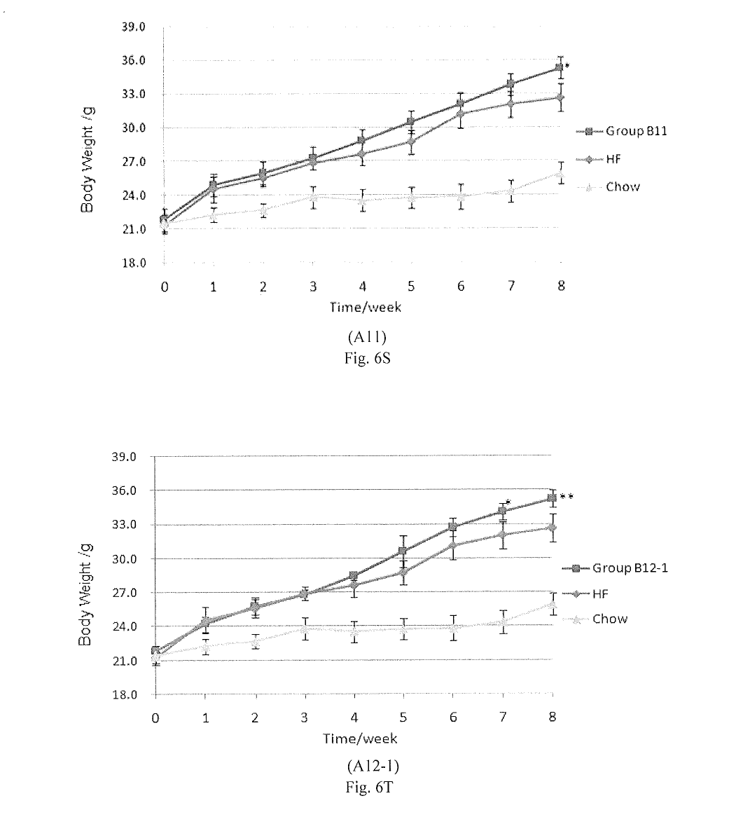

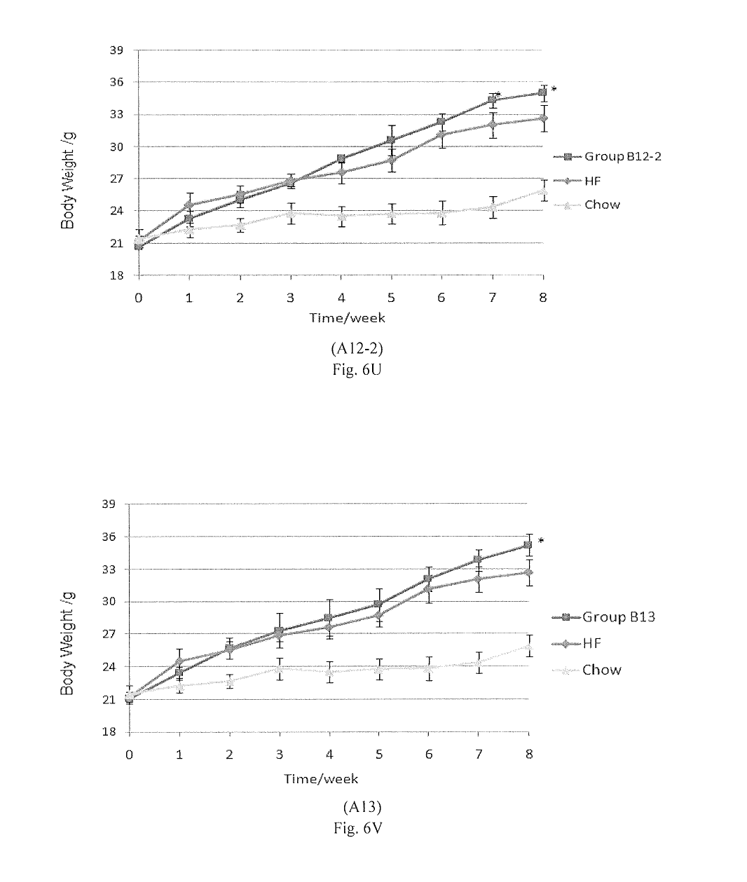

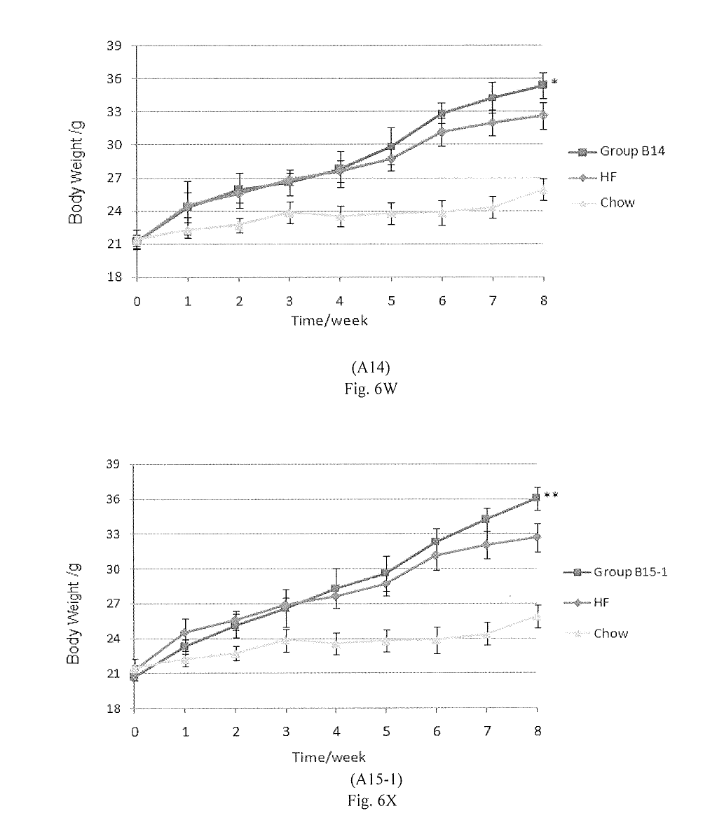

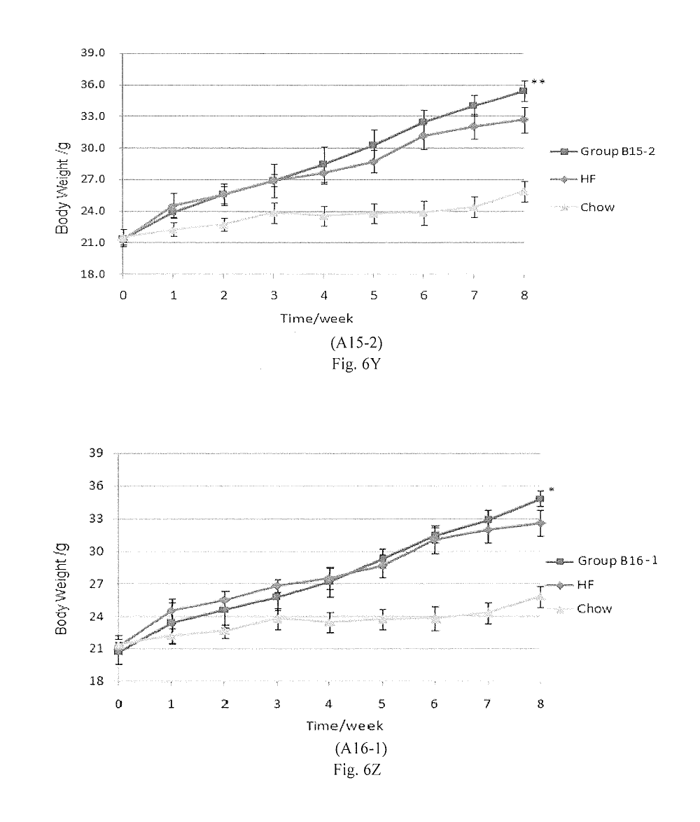

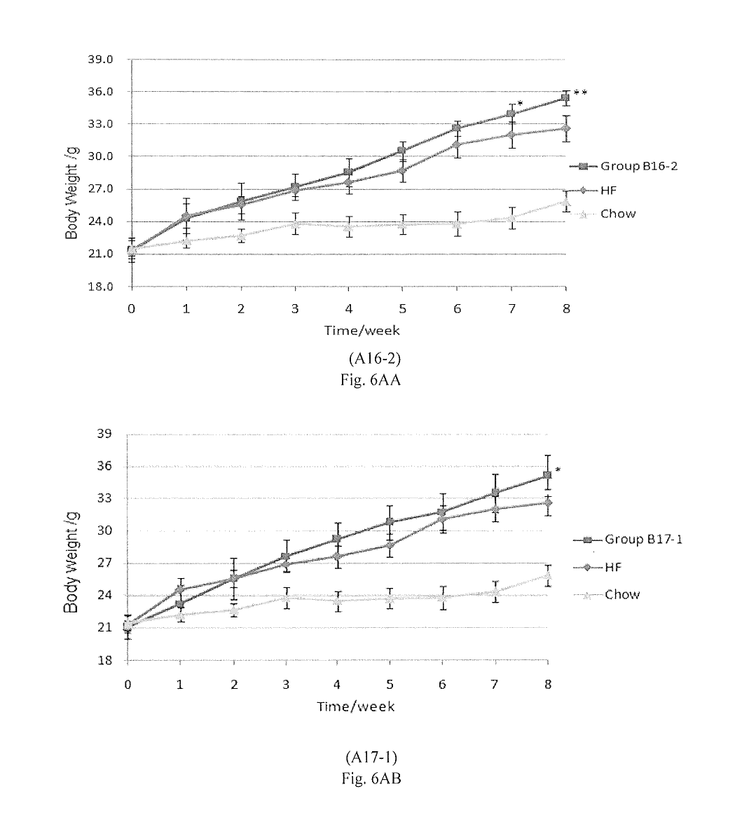

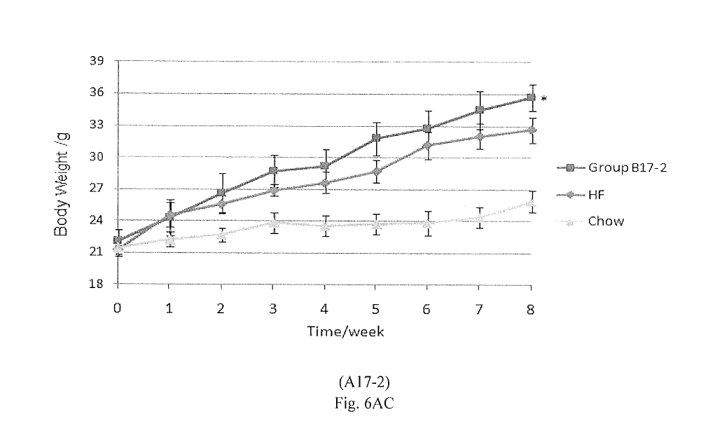

Referring to FIG. 2 to 4, the flow diagrams show the method to determine biomarkers related to T2D, comprising several main steps as follows:

### Example 3

### Identification of Biomarkers

3.1 Basic Analysis of Sequencing Data

After obtaining sequencing data from 145 samples of stage I, high quality reads were extracted by filtering low quality reads with ‘N’ base, adapter contamination or human DNA contamination from the Illumina raw data, totaling 378.4 Gb of high-quality data. On average, the proportion of high quality reads in all samples was about 98.1%, and the actual insert size of the PE library ranges from 313 bp to 381 bp.

3.2 Gene Catalogue Updating

Employing the same parameters that were used for building the MetaHIT gene catalogue (Junjie Qin, Ruiqiang Li, Jeroen Raes, et al. (2010) A human gut microbial gene catalogue established by metagenomic sequencing. Nature, 464:59-65, incorporated herein by reference), the inventors performed de novo assembly and gene prediction for 145 samples in stage I using SOAPdenovo v1.0642 and GeneMark v2.743, respectively. All predicted genes were aligned pairwise using BLAT and genes, of which over 90% of their length can be aligned to another one with more than 95% identity (no gaps allowed), were removed as redundancies, resulting in a non-redundant gene catalogue comprising of 2,088,328 genes. This gene catalogue from the Chinese samples was further combined with the previously constructed MetaHIT gene catalogue, by removing redundancies in the same manner. At last, the inventors obtained an updated gene catalogue with 4,267,985 predicted genes. 1,090,889 of these genes were uniquely assembled from the Chinese samples.

3.3 Taxonomic Assignment of Genes

Taxonomic assignment of the predicted genes was performed using an in-house pipeline. In the analysis, the inventors collected the reference microbial genomes from IMG database (v3.4), and then aligned all 4.2 million genes onto the reference genomes. Based on the comprehensive parameter exploration of sequence similarity across phylogenetic ranks by MetaHIT enterotype paper, the inventors used the 85% identity as the threshold for genus assignment (Arumugam, M. et al. Enterotypes of the human gut microbiome. Nature 473, 174-180, doi:10.1038/nature09944 (2011), incorporated herein by reference), as well as another threshold of 80% of the alignment coverage. For each gene, the highest scoring hit(s) above these two thresholds was chosen for the genus assignment. For the taxonomic assignment at the phylum level, the 65% identity was used instead. Here, 21.3% of the genes in the updated catalogue could be robustly assigned to a genus, which covered 26.4-90.6% (61.2% on average) of the sequencing reads in the 145 samples; the remaining genes were likely to be from currently undefined microbial species.

3.4 Functional Annotation

The inventors aligned putative amino acid sequences, which had been translated from the updated gene catalogue, against the proteins/domains in eggNOG (v3.0) and KEGG databases (release 59.0) using BLASTP (e-value 51e-5). Each protein was assigned to the KEGG orthologue group (KO) or eggNOG orthologue group (OG) by the highest scoring annotated hit(s) containing at least one HSP scoring over 60 bits. For the remaining genes without any annotation in eggNOG database, the inventors identified novel gene families based on clustering all-against-all BLASTP results using MCL with an inflation factor of 1.1 and a bit-score cutoff of 6045. Using this approach, the inventors identified 7,042 novel gene families (≧20 proteins) from the updated gene catalogue.

3.5 Quantification of Metagenome Content

3.5.1 Computation of Relative Gene Abundance

The high quality reads from each sample were aligned against the gene catalogue by SOAP2 using the criterion of “identity >90%”. In the sequence-based profiling analysis, only two types of alignments could be accepted: i). an entirety of a paired-end read can be mapped onto a gene with the correct insert-size; and ii). one end of the paired-end read can be mapped onto the end of a gene, only if the other end of read was mapped outside the genic region. In both cases, the mapped read was counted as one copy.

Then, for any sample 5, the inventors calculated the abundance as follows:

Step 1: Calculation of the copy number of each gene:

$${b}_{i} = \frac{{x}_{i}}{{L}_{i}}$$

Step 2: Calculation of the relative abundance of gene i

$${a}_{i} = \frac{{b}_{i}}{{Σ}_{j} {b}_{j}} = \frac{\frac{{x}_{i}}{{L}_{i}}}{{Σ}_{j} \frac{{x}_{j}}{{L}_{j}}}$$

aᵢ: The relative abundance of gene i in sample S.
Lᵢ: The length of gene i.
xᵢ: The times which gene i can be detected in sample S (the number of mapped reads).
bᵢ: The copy number of gene I in the sequenced data from sample s.

Based on gene relative profiles and the known taxonomic assignment and functional annotation of genes from above, one can sum up the gene relative abundances from the same species and from the same functional annotation respectively in order to obtain species relative abundance profiles and functions relative abundance profiles.

3.5.2 Estimation of Profiling Accuracy.

The inventors used the method developed by Audic and Claverie (Audic, S. & Claverie, J. M. The significance of digital gene expression profiles. Genome Res 7, 986-995 (1997), incorporated herein by reference) to assess the theoretical accuracy of the relative abundance estimates. Given that the inventors have observed vᵢ reads from gene i, as it occupied only a small part of total reads in a sample, the distribution of xᵢ is approximated well by a Poisson distribution. Let us denote N the total reads number in a sample, so N=Σᵢxᵢ. Suppose all genes are the same length, so the relative abundance value aᵢ of gene i simply is aᵢ=xᵢ/N. Then the inventors could estimate the expected probability of observing yᵢ reads from the same gene i, is given by the formula below,

$$P ( {a}_{i}^{′} \vert {a}_{i} ) = P ( {y}_{i} \vert {x}_{i} ) = \frac{( {x}_{i} + {y}_{i} ) !}{{x}_{i} ! {y}_{i} ! {2}^{( {x}_{i} + {y}_{i} + 1 )}}$$

Here, a′ᵢ=yᵢ/N is the relative abundance computed by yᵢ reads. Based on this formula, the inventors then made a simulation by setting the value of aᵢ from 0.0 to 1e-5 and N from 0 to 40 million, in order to compute the 99% confidence interval for a, and to further estimate the detection error rate (shown in FIG. 5).

3.5.3 Construction of Gene, KO, and OG Profile

The updated gene catalogue contains 4,267,985 non-redundant genes, which can be classified into 6,313 KOs (KEGG Orthologue) and 45,683 OGs (orthologue group in eggNOG, including 7,042 novel gene families). The inventors first removed genes, KOs or OGs that were present in less than 6 samples across all 145 samples in stage I. To reduce the dimensionality of the statistical analyses in MGWAS, in the construction of gene profile, the inventors identified highly correlated gene pairs and then subsequently clustered these genes using a straightforward hierarchical clustering algorithm. If the Pearson correlation coefficient between any two genes is >0.9, the inventors assigned an edge between these two genes. Then, the cluster A and B would not be clustered, if the total number of edges between A and B is smaller than |A|*|B|/3, where |A| and |B| are the sizes of A and B, respectively. Only the longest gene in a gene linkage group was selected to represent this group, yielding a total of 1,138,151 genes. These 1,138,151 genes and their associated measures of relative abundance in 145 stage I samples were used to establish the gene profile for the association study.

For the KO profile, the inventors utilized the gene annotation information of the original U.S. Pat. No. 4,267,985 genes and summed the relative abundance of genes from the same KO. This gross relative abundance was taken as the content of this KO in a sample to generate the KO profile of 145 samples. The OG profile was constructed using the same method used for KO profile.

3.6 Enterotypes Identification

The relative abundance of a genus was estimated by the same method used in construction of KO profile, and then was used for identifying enterotypes from the Chinese samples. The inventors used the same identification method as described in the original paper of enterotypes (Arumugam, M. et al. Enterotypes of the human gut microbiome. Nature 473, 174-180, doi:10.1038/nature09944 (2011), incorporated herein by reference). In the study, samples were clustered using Jensen-Shannon distance.

$$JSD ( P \vert \vert D ) = \frac{1}{2} D ( P \vert \vert M ) + \frac{1}{2} D ( Q \vert \vert M )$$

$$in which \text{:}$$

$$M = \frac{1}{2} ( P + Q )$$

$$D ( P \vert \vert M ) = {Σ}_{i} P ( i ) ln \frac{P ( i )}{M ( i )}$$

$$D ( Q \vert \vert M ) = {Σ}_{i} Q ( i ) ln \frac{Q ( i )}{M ( i )}$$

P (i) and Q (i) are the relative abundances of gene i in sample P, Q respectively. Enterotype of each sample can be validated by the same method on OG/KO relative profile.

3.7 Statistical Analysis of MGWAS

3.7.1 PERMANOVA

In the study, Permutational Multivariate Analysis Of Variance (PERMANOVA, McArdle, B. H. & Anderson, M. J. Fitting Multivariate Models to Community Data: A Comment on Distance-Based Redundancy Analysis. Ecology 82, 290-297 (2001), incorporated herein by reference) was used to assess the effect of each covariate including enterotype, T2D, age, gender and BMI, on four types of profiles. The inventors performed the analysis using the method implemented in R package—“vegan” (Zapala, M. A. & Schork, N. J. Multivariate regression analysis of distance matrices for testing associations between gene expression patterns and related variables. Proceedings of the National Academy of Sciences of the United States of America 103, 19430-19435, doi:10.1073/pnas.0609333103 (2006), incorporated herein by reference), and the permuted P-value was obtained by 10,000 times permutations.

| | | | P-values (top 20 |
|---|---|---|---|
| | No. | P-values (original | principal components |
| Variables | subjects | gene profile) | in original gene profile) |
| Enterotypes | 3 | 0.0001 | 0.0001 |
| T2D | 2 | 0.0305 | 0.0004 |
| BMI | 255 | 0.3308 | 0.1851 |
| Gender | 2 | 0.2129 | 0.1326 |
| Age | 63 | 0.2030 | 0.1044 |

3.7.2 Population Stratifications.

To correct population stratifications of the data, the inventors used a modified version of the EIGENSTRAT method (Price, A. L. et al. Principal components analysis corrects for stratification in genome-wide association studies. Nature genetics 38, 904-909, doi:10.1038/ng1847 (2006), incorporated herein by reference) allowing the use of covariance matrices estimated from abundance levels instead of genotypes. However, as much of the signal in the data might be driven by the combined effect of many genes and not by just a few genes as assumed in GWAS studies, the inventors modified the method further by replacing each PC axis with the residuals of this PC axis from a regression to T2D. The number of PC axes of EIGENSTAT was determined by Tracy-Widom test at a significance level of P<0.0551.

3.7.3 Statistical Hypothesis Test on Profiles

In stage I, to identify the association between the metagenome profile and T2D, a two-tailed Wilcoxon rank-sum test was used in the profiles that were adjusted for non-T2D-related population stratifications. Then, while examining the stage I markers in stage II, a one-tailed Wilcoxon rank-sum test was used instead. Because the T2D is the primary factor impacting on the profile of examined gene markers in stage II, we didn't adjust the population stratification for these genes.

3.7.4 Estimating the False Discovery Rate (FDR) and the Power

Instead of a sequential P-value rejection method, we applied the “q value” method proposed in a previous study (Storey, J. D. A direct approach to false discovery rates. Journal of the Royal Statistical Society—Series B: Statistical Methodology 64, 479-498 (2002), incorporated herein by reference) to estimate the false discovery rate (FDR). In our MWAS, the statistical hypothesis tests were performed on a large number of features of the gene, KO, OG and genus profiles. Given that a FDR was obtained by the q value method 53, we estimated the power P, for a given p-value threshold by the formula below,

$${P}_{e} = \frac{{N}_{e} ( 1 - {FDR}_{e} )}{N ( 1 - {π}_{0} )}$$

Here, π₀ is the proportion of null distribution P-values among all tested hypotheses; Nₑ is the number of P-values that were less than the P-value threshold; N is the total number of all tested hypotheses; FDRₑ is the estimated false discovery rate under the P-value threshold.

3.8 Selection of Biomarkers

In stage I the inventors use two-side Wilcox test based on population-adjusted stage I gene and functions (KO and OG) relative abundance profile and the inventors adjust the multiple test by estimating the false discovery rate (FDR). Finally the gene passing the test was the biomarkers. At last, the inventors use a clustering method to cluster the genes into species biomarkers (called MLG). And the inventors test the gene, functions (KO and OG), species biomarkers by Student T test. The p-value of each biomarkers are summarized in Table 2.

To reduce and structurally organize the abundant metagenomic data and to enable us to make a taxonomic description, the inventors devised the generalized concept of Metagenomic Linkage Group (MLG) in lieu of a species concept for a metagenome. Here a MLG is defined as a group of genetic material in a metagenome that is likely physically linked as a unit rather than being independently distributed; this allowed us to avoid the need to completely determine the specific microbial species present in the metagenome, which is important given there are a large number of unknown organisms and that there is frequent lateral gene transfer (LGT) between bacteria. Using the gene profile, the inventors defined and identified a MLG as a group of genes that co-exists among different individual samples and has a consistent abundance level and taxonomic assignment.

3.9 Identification of Metagenomic Linkage Group (MLG)

3.9.1 the Clustering Method for Identifying MLG

In the present study, the inventors devised a concept of metagenomic linkage group (MLG), which could facilitate the taxonomic description of metagenomic data from whole-genome shotgun sequencing. To identify MLG from the set of T2D-associated gene markers, the inventors developed an in-house software that comprises three steps as indicated below:

Step 1: The original set of T2D-associated gene markers was taken as initial subclusters of genes. It should be noted that in the establishment of the gene profile the inventors had constructed gene linkage groups to reduce the dimensionality of the statistical analysis. Accordingly, all genes from a gene linkage group were considered as one subcluster.
Step 2: The inventors applied the Chameleon algorithm (Karypis, G & Kumar, V. Chameleon: hierarchical clustering using dynamic modeling. Computer 32, 68-75 (1999), incorporated herein by reference) to combine the subclusters exhibiting a minimal similarity of 0.4 using dynamic modeling technology and basing selection on both interconnectivity and closeness 54. The similarity here is defined by the product of interconnectivity and closeness (the inventors used this definition in the whole analysis of MLG identification). The inventors term these clusters semi-clusters.
Step 3: To further merge the semi-clusters established in step 2, in this step, the inventors first updated the similarity between any two semi-clusters, and then performed a taxonomic assignment for each semi-cluster (see the method below). Finally, two or more semi-clusters would be merged into a MLG if they satisfied both of the following two requirements: a) the similarity values between the semi-clusters were >0.2; and b) all these semi-clusters were assigned from the same taxonomy lineage.

3.9.2 Taxonomic Assignment for a MLG

All genes from a MLG were aligned to the reference microbial genomes (IMG database, v3.4) at the nucleotide level (by BLASTN) and the NCBI-nr database (February 2012) at the protein level (by BLASTP). The alignment hits were filtered by both the e-value (<1×10-10 at the nucleotide level and <1×10-5 at the protein level) and the alignment coverage (>70% of a query sequence). From the alignments with the reference microbial genomes, the inventors obtained a list of well-mapped bacterial genomes for each MGL group and ordered these bacterial genomes according to the proportion of genes that could be mapped onto the bacterial genome, as well as the average identity of the alignments. The taxonomic assignment of a MLG was determined by the following principles: 1) if more than 90% of genes in this MLG can be mapped onto a reference genome with a threshold of 95% identity at the nucleotide level, the inventors considered this particular MLG to originate from this known bacterial species; 2) if more than 80% of genes in this MLG can be mapped onto a reference genome with a threshold of 85% identity at the both nucleotide and protein levels, the inventors considered this MLG to originate from the same genus of the matched bacterial species; 3) if the 16S sequences can be identified from the assembly result of a MLG, the inventors performed the phylogenetic analysis by RDP-classifier55 (bootstrap value >0.80) (Wang, Q., Garrity, G M., Tiedje, J. M. & Cole, J. R. Naive Bayesian classifier for rapid assignment of rRNA sequences into the new bacterial taxonomy. Appl Environ Microbiol 73, 5261-5267, doi:AEM.00062-07 [pii]10.1128/AEM.00062-07 (2007), incorporated herein by reference) and then defined the taxonomic assignment for the MLG if the phylotype from 16S sequences was consistent with that from genes.

3.9.3 Advanced-Assembly for a MLG

To reconstruct the potential bacterial genomes, the inventors designed an additional process of advanced-assembly for each MLG, which was implemented in four steps.

Step 1: Taking the genes from a MLG as a seed, the inventors identified samples that contain the seed with the highest abundance among all samples, and then selected the paired-end reads from these samples that could be mapped onto the seed (including the paired-end read that only one end could be mapped). The lower limit of the coverage of these paired-end reads is 50× in no more than 5 samples, which is computed by dividing the total size of selected reads by the total length of the seed.
Step 2: A de novo assembly was performed on the selected reads in step 1 by using the SOAPdenovo with the same parameters used for the construction of the gene catalogue.
Step 3: To identify and remove the mis-assembled contigs probably caused by contaminated reads, the inventors applied a composition-based binning method. Contigs whose GC content value and sequencing depth value were distinct from the other contigs of the assembly result were removed, as they might be wrongly assembled due to various reasons.
Step 4: Taking the final assembly result from step 3 as a seed, the inventors repeated the procedure from step 2 until that there were no further distinct improvements of the assembly (in detail, the increment of total contig size was less than 5%).

3.10 MLG-Based Analysis

3.10.1 Validation of MLG Methods

The performance of the MLG identification methods was evaluated by following steps: 1). In the quantified gene result, the rarely present genes (present in <6 samples) were filtered at first; 2) Based on the taxonomic assignment result in the updated gene catalogue, the inventors identified a set of gut bacterial species by the criteria of containing 1,000-5,000 unique mapped genes, with the similarity threshold of 95%. In this step, the inventors manually removed the redundant strains in one species and also discarded the genes that could be mapped onto more than one species. Ultimately, 130,065 genes from 50 gut bacterial species were identified as a test set for validating the MLG method; 3). The standard MLG method described above was performed on the test set. For each MLG, the inventors computed the percentage of genes that were not from the major species as an error rate (namely % gene, shown in Table 7).

3.10.2 Relative Abundance of a MLG

The inventors estimated the relative abundance of a MLG in all samples by using the relative abundance values of genes from this MLG For this MLG, the inventors first discarded genes that were among the 5% with the highest and lowest relative abundance, respectively, and then fitted a Poisson distribution to the rest. The estimated mean of the Poisson distribution was interpreted as the relative abundance of this MLG. At last, the profile of MLGs among all samples was obtained for the following analyses.

### Example 4

### A Two-Stage Validation

4.1 Data Analysis

The inventors repeat Example 1 and Example 2 steps to get sequenced data and repeat Example 3 steps to get gene, functions and species relative profile with the use of 199 samples in stage II.

4.2 Validation of Biomarkers

In stage I the inventors use two-side Wilcox test based on population-adjusted stage I gene and functions (KO and OG) relative abundance profile and In stage II the inventors use one-side Wilcox test based on origin gene and functions (KO and OG) relative abundance profile and the side is determined by stage I genes direction. And the inventors adjust the multiple test by estimating the false discovery rate (FDR). Finally the gene passing the test was the biomarkers. At last, the inventors use a clustering method to cluster the genes into species biomarkers (called MLG). And the inventors test the gene, functions (KO and OG), species biomarkers by Student T test. The p-value of each biomarkers are summarized in Table 2.

The inventors next control for the false discovery rate (FDR) in the stage 11 analysis, and define a total of 52,484 T2D-associated gene markers from these genes corresponding to a FDR of 2.5% (Stage II P value <0.01). The inventors apply the same two-stage analysis using the KO and OG profiles and identified a total of 1,345 KO markers (Stage II P<0.05 and 4.5% FDR) and 5,612 OG markers (Stage II P<0.05 and 6.6% FDR) that are associated with T2D.

TABLE 2
Species makers

| Enrichment | | | |
|---|---|---|---|
| (direction) | MLG^{a}ID | P-values^{b} (stage I) | P-values^{b} (stage II) |
| T2D group | T2D-154 | 0.001347368 | 0.000254046 |
| enrichment | T2D-140 | 0.000397275 | 0.002849677 |
| | T2D-139 | 0.001328967 | 0.000211459 |
| | T2D-11 | 4.16065E−08 | 7.58308E−05 |
| | T2D-5 | 4.21047E−05 | 1.97056E−06 |
| | T2D-80 | 0.000129893 | 1.40862E−05 |
| | T2D-57 | 4.00759E−07 | 2.20525E−05 |
| | T2D-15 | 4.74327E−05 | 0.00029675 |
| | T2D-1 | 0.000601047 | 0.003604634 |
| | T2D-7 | 0.000601047 | 0.000279527 |
| | T2D-137 | 6.70507E−07 | 0.001204531 |
| control group | Con-107 | 1.12113E−07 | 0.001826862 |
| enrichment | Con-112 | 0.006389079 | 0.00019943 |
| | Con-129 | 0.003274757 | 0.001001054 |
| | Con-166 | 3.79947E−05 | 0.000193721 |
| | Con-121 | 6.10793E−05 | 4.89846E−06 |
| | Con-113 | 0.000284629 | 0.000972347 |

^{a}MLG: Metagenomic Linkage Group, defined as candidate species.
^{b}The null hypothesis is that T2D groups don't differ from Control groups on the MLG, P value (P value <0.05, considering as significant) means the probability of obtaining a test statistic at least as extreme as the one that was actually observed, assuming that the null hypothesis is true.

4.3 Prediction Analysis of Species Makers

4.3.10 One Species Prediction System

Using the species relative abundances as the risk score, the inventors estimate the AUC (Michael J. Pencina, Ralph B. D'Agostino Sr, Ralph B. D'Agostino Jr, et al. Evaluating the added predictive ability of a new marker: From area under the ROC curve to reclassification and beyond. Statistics in medicine, 2008, 27(2): 157-172, incorporated herein by reference). The larger the AUC is, the more powerful the prediction ability on T2D disease is. For each species, the inventors can estimate an AUC and its best cutoff where the sum of the prediction sensitivity and specificity reaches its maximum.

Detail of the cutoff: for a species, the inventors first sort the samples' relative abundances. The inventors sequentially treat each relative abundance as the candidate cutoff and estimate its sensitivity and specificity. So the inventors can get the best cutoff on the maximal sum of the prediction sensitivity and specificity. For beneficial species, if the test sample's relative abundance is less than the best cutoff then the inventors predict the test sample is in disease condition. For harmful species, if the test sample's relative abundance is larger than the best cutoff then the inventors predict the test sample is in disease condition. See Table 3.

Sensitivity (also called recall rate in some fields) measures the proportion of actual positives which are correctly identified as such (e.g. the percentage of sick people who are correctly identified as having the condition). Specificity measures the proportion of negatives which are correctly identified (e.g. the percentage of healthy people who are correctly identified as not having the condition).

TABLE 3
AUC and CUTOFF of species markers

| | Enrichment^{c} | | | | |
|---|---|---|---|---|---|
| MLG ID | (direction) | cutoff | AUC | sensitivity | specificity |
| T2D-11 | 1 | 0.103658 | 0.618 | 0.541176 | 0.66092 |
| T2D-137 | 1 | 0.498151 | 0.585 | 0.423529 | 0.729885 |
| T2D-139 | 1 | 1.553228 | 0.617 | 0.5 | 0.701149 |
| T2D-140 | 1 | 0.49045 | 0.571 | 0.423529 | 0.735632 |
| T2D-154 | 1 | 8.95E−05 | 0.604 | 0.411765 | 0.798851 |
| T2D-15 | 1 | 0.00508 | 0.589 | 0.670588 | 0.494253 |
| T2D-1 | 1 | 0.098314 | 0.526 | 0.076471 | 0.977011 |
| T2D-57 | 1 | 0.015788 | 0.647 | 0.523529 | 0.701149 |
| T2D-5 | 1 | 0.000673 | 0.651 | 0.688235 | 0.563218 |
| T2D-7 | 1 | 0.046154 | 0.604 | 0.523529 | 0.655172 |
| T2D-80 | 1 | 0.003178 | 0.655 | 0.682353 | 0.586207 |
| Con-107 | 0 | 0.34953 | 0.656 | 0.652941 | 0.637931 |
| Con-112 | 0 | 0.059392 | 0.606 | 0.529412 | 0.632184 |
| Con-113 | 0 | 0.36604 | 0.646 | 0.641176 | 0.614943 |
| Con-121 | 0 | 0.06585 | 0.67 | 0.688235 | 0.568966 |
| Con-129 | 0 | 0.663083 | 0.618 | 0.658824 | 0.557471 |
| Con-166 | 0 | 0.001912 | 0.67 | 0.5 | 0.781609 |

^{c}1 represents T2D group enrichment and harmful marker; 0 represents control group enrichment and beneficial marker.

4.3.2 Global Prediction System.

Above the inventors have built a prediction system on one species, below the inventors build a system based on a synthetical score that combing all the species biomarkers to predict test sample's disease risk. The system is that the inventors estimate a best cutoff by same ROC method above on the synthetical score (shown in Table 5). At the condition that disease group average synthetical score are larger than the control group (the inventors name this condition as direction 1), if a test sample synthetical score is larger than the best cutoff then it is treated as in disease status else it is healthy. On the contrary at the condition that disease group average synthetical score are less than the control group (the inventors name this condition as direction 0), if a test sample synthetical score is less than the best cutoff then it is treated as in disease status else it is healthy. Prediction performance are summarized in Table 4 and 5.

Details of synthetical score: the inventors build a score matrix as the same size as the species profile. For each species and each sample, the inventors assign a score I if the sample is predict to be in disease status based on the one species prediction system the inventors have built above and assign a score 0 if the sample is predict to be healthy. The inventors sum the scores in the score matrix for each sample as the synthetical score.

TABLE 4
Synthetical score (cutoff)

| synthetical | | | | |
|---|---|---|---|---|
| score | | | | |
| (cutoff) | AUC | sensitivity | specificity | direction |
| 6 | 0.77 | 0.782353 | 0.54023 | 1 |

TABLE 5
Prediction

| Sample | | synthet- | Samples ID | | synthet- |
|---|---|---|---|---|---|
| ID (T2D | T2D | ical | (control | T2D | ical |
| samples | prediction^{d} | score | samples) | prediction^{d} | score |
| DLF001 | 1 | 12 | NLF001 | 0 | 2 |
| DLF002 | 1 | 10 | NLF002 | 0 | 5 |
| DLF003 | 0 | 5 | NLF005 | 0 | 1 |
| DLF004 | 1 | 8 | NLF006 | 0 | 3 |
| DLF005 | 0 | 4 | NLF007 | 0 | 4 |
| DLF006 | 1 | 11 | NLF008 | 1 | 13 |
| DLF007 | 1 | 11 | NLF009 | 0 | 1 |
| DLF008 | 1 | 12 | NLF010 | 0 | 6 |
| DLF009 | 1 | 16 | NLF011 | 0 | 2 |
| DLF010 | 1 | 7 | NLF012 | 0 | 4 |
| DLF012 | 1 | 9 | NLF013 | 0 | 1 |
| DLF013 | 1 | 13 | NLF014 | 1 | 12 |
| DLF014 | 0 | 6 | NLF015 | 1 | 8 |
| DLM001 | 1 | 9 | NLM001 | 1 | 7 |
| DLM002 | 1 | 7 | NLM002 | 1 | 7 |
| DLM003 | 1 | 10 | NLM003 | 1 | 12 |
| DLM004 | 1 | 9 | NLM004 | 0 | 2 |
| DLM005 | 1 | 8 | NLM005 | 1 | 9 |
| DLM006 | 1 | 7 | NLM006 | 1 | 7 |
| DLM007 | 1 | 12 | NLM007 | 1 | 9 |
| DLM008 | 1 | 9 | NLM008 | 0 | 5 |
| DLM009 | 1 | 11 | NLM009 | 0 | 0 |
| DLM010 | 1 | 7 | NLM010 | 1 | 8 |
| DLM011 | 1 | 10 | NLM015 | 1 | 8 |
| DLM012 | 1 | 12 | NLM016 | 0 | 5 |
| DLM013 | 1 | 13 | NLM017 | 1 | 14 |
| DLM014 | 1 | 7 | NLM021 | 0 | 3 |
| DLM015 | 1 | 12 | NLM022 | 0 | 1 |
| DLM016 | 1 | 7 | NLM023 | 1 | 13 |
| DLM017 | 0 | 4 | NLM024 | 1 | 10 |
| DLM018 | 0 | 5 | NLM025 | 0 | 4 |
| DLM019 | 0 | 5 | NLM026 | 0 | 3 |
| DLM020 | 1 | 8 | NLM027 | 1 | 9 |
| DLM021 | 1 | 8 | NLM028 | 0 | 5 |
| DLM022 | 1 | 14 | NLM029 | 0 | 2 |
| DLM023 | 1 | 12 | NLM031 | 0 | 5 |
| DLM024 | 1 | 14 | NLM032 | 0 | 1 |
| DLM027 | 0 | 6 | NOF001 | 0 | 6 |
| DLM028 | 1 | 9 | NOF002 | 1 | 8 |
| DOF002 | 1 | 8 | NOF004 | 0 | 2 |
| DOF003 | 1 | 7 | NOF005 | 1 | 7 |
| DOF004 | 1 | 10 | NOF006 | 1 | 9 |
| DOF006 | 1 | 12 | NOF007 | 0 | 5 |
| DOF007 | 1 | 12 | NOF008 | 1 | 10 |
| DOF008 | 0 | 6 | NOF009 | 1 | 13 |
| DOF009 | 1 | 7 | NOF010 | 1 | 12 |
| DOF010 | 1 | 15 | NOF011 | 0 | 6 |
| DOF011 | 0 | 3 | NOF012 | 1 | 10 |
| DOF012 | 1 | 11 | NOF013 | 1 | 7 |
| DOF013 | 1 | 8 | NOF014 | 0 | 6 |
| DOF014 | 0 | 6 | NOM001 | 0 | 3 |
| DOM001 | 1 | 11 | NOM002 | 0 | 6 |
| DOM003 | 0 | 5 | NOM004 | 1 | 12 |
| DOM005 | 1 | 12 | NOM005 | 0 | 3 |
| DOM008 | 1 | 15 | NOM007 | 0 | 0 |
| DOM010 | 1 | 9 | NOM008 | 1 | 8 |
| DOM012 | 1 | 10 | NOM009 | 0 | 2 |
| DOM013 | 1 | 7 | NOM010 | 0 | 4 |
| DOM014 | 1 | 7 | NOM012 | 0 | 5 |
| DOM015 | 1 | 7 | NOM013 | 0 | 4 |
| DOM016 | 1 | 10 | NOM014 | 1 | 8 |
| DOM017 | 0 | 4 | NOM015 | 1 | 8 |
| DOM018 | 0 | 2 | NOM016 | 0 | 3 |
| DOM019 | 1 | 8 | NOM017 | 0 | 2 |
| DOM020 | 0 | 6 | NOM018 | 0 | 0 |
| DOM021 | 1 | 9 | NOM019 | 0 | 5 |
| DOM022 | 1 | 12 | NOM020 | 0 | 4 |
| DOM023 | 0 | 6 | NOM022 | 0 | 4 |
| DOM024 | 1 | 9 | NOM023 | 1 | 8 |
| DOM025 | 1 | 13 | NOM025 | 0 | 5 |
| DOM026 | 1 | 8 | NOM026 | 0 | 1 |
| T2D. 016 | 1 | 11 | NOM027 | 1 | 8 |
| T2D. 017 | 1 | 9 | NOM028 | 1 | 13 |
| T2D. 018 | 0 | 6 | NOM029 | 0 | 5 |
| T2D. 019 | 1 | 16 | CON. 016 | 0 | 6 |
| T2D. 020 | 1 | 11 | CON. 032 | 1 | 7 |
| T2D. 021 | 1 | 9 | CON. 033 | 0 | 3 |
| T2D. 071 | 0 | 4 | CON. 034 | 0 | 5 |
| T2D. 022 | 1 | 8 | CON. 017 | 0 | 3 |
| T2D. 046 | 1 | 10 | CON. 035 | 1 | 8 |
| T2D. 001 | 1 | 11 | CON. 036 | 1 | 8 |
| T2D. 047 | 1 | 15 | CON. 037 | 0 | 1 |
| T2D. 048 | 1 | 9 | CON. 001 | 0 | 6 |
| T2D. 049 | 1 | 11 | CON. 038 | 0 | 0 |
| T2D. 023 | 0 | 4 | CON. 018 | 0 | 4 |
| T2D. 024 | 0 | 3 | CON. 081 | 0 | 4 |
| T2D. 050 | 1 | 12 | CON. 082 | 0 | 1 |
| T2D. 025 | 0 | 3 | CON. 019 | 1 | 9 |
| T2D. 072 | 1 | 12 | CON. 039 | 1 | 12 |
| T2D. 073 | 0 | 3 | CON. 002 | 0 | 6 |
| T2D. 051 | 1 | 14 | CON. 083 | 0 | 5 |
| T2D. 026 | 1 | 14 | CON. 084 | 0 | 2 |
| T2D. 074 | 1 | 14 | CON. 003 | 0 | 5 |
| T2D. 075 | 1 | 12 | CON. 040 | 0 | 3 |
| T2D. 076 | 1 | 15 | CON. 041 | 1 | 9 |
| T2D. 052 | 0 | 4 | CON. 042 | 1 | 7 |
| T2D. 077 | 1 | 12 | CON. 043 | 0 | 6 |
| T2D. 053 | 0 | 2 | CON. 004 | 1 | 9 |
| T2D. 002 | 1 | 9 | CON. 044 | 0 | 3 |
| T2D. 078 | 1 | 10 | CON. 085 | 0 | 5 |
| T2D. 054 | 1 | 8 | CON. 020 | 0 | 3 |
| T2D. 079 | 1 | 8 | CON. 045 | 1 | 12 |
| T2D. 080 | 1 | 14 | CON. 046 | 1 | 8 |
| T2D. 003 | 1 | 10 | CON. 086 | 1 | 8 |
| T2D. 055 | 1 | 8 | CON. 087 | 0 | 3 |
| T2D. 081 | 1 | 9 | CON. 047 | 0 | 4 |
| T2D. 056 | 1 | 7 | CON. 088 | 1 | 11 |
| T2D. 082 | 1 | 7 | CON. 005 | 0 | 6 |
| T2D. 028 | 1 | 9 | CON. 006 | 1 | 9 |
| T2D. 083 | 1 | 14 | CON. 089 | 0 | 6 |
| T2D. 029 | 0 | 5 | CON. 048 | 1 | 13 |
| T2D. 057 | 1 | 12 | CON. 090 | 0 | 4 |
| T2D. 004 | 0 | 6 | CON. 007 | 1 | 13 |
| T2D. 058 | 1 | 9 | CON. 091 | 1 | 10 |
| T2D. 084 | 1 | 9 | CON. 008 | 1 | 7 |
| T2D. 059 | 1 | 9 | CON. 049 | 0 | 6 |
| T2D. 030 | 0 | 6 | CON. 092 | 1 | 8 |
| T2D. 005 | 1 | 7 | CON. 050 | 1 | 11 |
| T2D. 031 | 1 | 11 | CON. 009 | 1 | 7 |
| T2D. 085 | 1 | 8 | CON. 051 | 1 | 8 |
| T2D. 086 | 0 | 1 | CON. 093 | 0 | 2 |
| T2D. 006 | 0 | 5 | CON. 052 | 1 | 9 |
| T2D. 007 | 1 | 13 | CON. 053 | 1 | 9 |
| T2D. 060 | 1 | 14 | CON. 054 | 0 | 6 |
| T2D. 087 | 1 | 11 | CON. 095 | 0 | 2 |
| T2D. 008 | 1 | 11 | CON. 021 | 1 | 7 |
| T2D. 088 | 1 | 9 | CON. 055 | 1 | 11 |
| T2D. 009 | 0 | 6 | CON. 022 | 0 | 4 |
| T2D. 089 | 1 | 13 | CON. 096 | 1 | 9 |
| T2D. 036 | 1 | 13 | CON. 097 | 1 | 7 |
| T2D. 039 | 1 | 7 | CON. 023 | 1 | 9 |
| T2D. 090 | 1 | 14 | CON. 098 | 0 | 6 |
| T2D. 091 | 1 | 12 | CON. 056 | 0 | 5 |
| T2D. 062 | 0 | 4 | CON. 099 | 0 | 2 |
| T2D. 063 | 1 | 11 | CON. 057 | 0 | 2 |
| T2D. 040 | 1 | 7 | CON. 101 | 0 | 2 |
| T2D. 092 | 1 | 12 | CON. 058 | 1 | 7 |
| T2D. 064 | 0 | 6 | CON. 059 | 0 | 0 |
| T2D. 093 | 0 | 5 | CON. 060 | 1 | 10 |
| T2D. 010 | 1 | 11 | CON. 061 | 0 | 0 |
| T2D. 094 | 0 | 5 | CON. 104 | 0 | 1 |
| T2D. 011 | 0 | 6 | CON. 062 | 0 | 4 |
| T2D. 041 | 0 | 6 | CON. 010 | 0 | 5 |
| T2D. 096 | 1 | 14 | CON. 063 | 0 | 1 |
| T2D. 065 | 1 | 13 | CON. 064 | 0 | 5 |
| T2D. 097 | 0 | 2 | CON. 105 | 0 | 1 |
| T2D. 066 | 1 | 9 | CON. 065 | 0 | 5 |
| T2D. 098 | 1 | 9 | CON. 066 | 0 | 1 |
| T2D. 012 | 1 | 11 | CON. 011 | 0 | 3 |
| T2D. 042 | 1 | 8 | CON. 067 | 1 | 10 |
| T2D. 013 | 1 | 10 | CON. 068 | 0 | 4 |
| T2D. 099 | 1 | 8 | CON. 069 | 0 | 5 |
| T2D. 100 | 1 | 11 | CON. 012 | 0 | 4 |
| T2D. 101 | 1 | 10 | CON. 070 | 0 | 1 |
| T2D. 102 | 1 | 8 | CON. 106 | 0 | 4 |
| T2D. 067 | 1 | 12 | CON. 071 | 0 | 3 |
| T2D. 103 | 1 | 13 | CON. 026 | 0 | 1 |
| T2D. 104 | 1 | 9 | CON. 072 | 0 | 0 |
| T2D. 043 | 1 | 12 | CON. 107 | 0 | 0 |
| T2D. 105 | 1 | 10 | CON. 073 | 1 | 8 |
| T2D. 044 | 1 | 8 | CON. 027 | 0 | 5 |
| T2D. 106 | 0 | 0 | CON. 074 | 0 | 6 |
| T2D. 014 | 1 | 10 | CON. 075 | 0 | 2 |
| T2D. 068 | 1 | 12 | CON. 028 | 0 | 2 |
| T2D. 107 | 1 | 8 | CON. 029 | 0 | 3 |
| T2D. 069 | 1 | 7 | CON. 013 | 0 | 6 |
| T2D. 045 | 1 | 16 | CON. 076 | 0 | 1 |
| T2D. 070 | 1 | 14 | CON. 014 | 0 | 1 |
| T2D. 015 | 1 | 13 | CON. 077 | 0 | 4 |
| T2D. 108 | 1 | 11 | CON. 078 | 0 | 3 |
| | | | CON. 015 | 0 | 4 |
| | | | CON. 079 | 1 | 8 |
| | | | CON. 080 | 1 | 11 |
| | | | CON. 031 | 0 | 1 |

^{d}1 represents that the sample is predicted to be T2D; 0 represents that the sample is predicted to be non-T2D.

### Example 5

### Rebuilt Microbial Genomes Associated with Diseases

5.1 Advanced-Assembly

Use the method in Example 3 to conduct MLG advanced-assembly rebuilt microbial genomes associated with diseases (results shown in Table 6).

TABLE 6
MLG Advanced-assembly

| MLG ID | Assembled size (bp) |
|---|---|
| T2D-154 | 1,459,858 |
| T2D-140 | 306,933 |
| T2D-139 | 4,076,917 |
| T2D-11 | 5,461,429 |
| T2D-5 | 5,685,283 |
| T2D-80 | 3,343,701 |
| T2D-57 | 2,235,135 |
| T2D-15 | 4,343,101 |
| T2D-1 | 1,147,560 |
| T2D-7 | 1,475,127 |
| T2D-137 | 360,515 |
| Con-107 | 2,425,544 |
| Con-112 | 625,210 |
| Con-129 | 2,763,410 |
| Con-166 | 300,056 |
| Con-121 | 3,263,915 |
| Con-113 | 912,962 |

5.2 Identification of Microbial Genomes

Use the method in Example 3 to conduct MLG taxonomic assignment based on the obtained microbial genomes (results shown in Table 7).

TABLE 7
MLG Taxonomic assignment

| | MLG | Number | Taxonomy assignment | | |
|---|---|---|---|---|---|
| Enrichment | ID | of genes | (level) | % genes^{e} | similarity^{f} |
| T2D group | T2D-154 | 337 | Akkermansia muciniphila | 97.92 | 98.17 ± 0.09 |
| enrichment | T2D-140 | 148 | Bacteroides intestinalis | 89.19 | 98.20 ± 0.15 |
| | T2D-139 | 3,386 | Bacteroides sp. 20_3 | 94.60 | 99.29 ± 0.01 |
| | T2D-11 | 5,113 | Clostridium bolteae | 96.87 | 99.39 ± 0.02 |
| | T2D-5 | 2,378 | Clostridium hathewayi | 96.93 | 99.31 ± 0.03 |
| | T2D-80 | 2,381 | Clostridium ramosum | 95.38 | 99.81 ± 0.01 |
| | T2D-57 | 821 | Clostridium sp. HGF2 | 97.69 | 99.59 ± 0.03 |
| | T2D-15 | 2,492 | Clostridium symbiosum | 95.63 | 99.58 ± 0.01 |
| | T2D-1 | 949 | Desulfovibrio sp. 3_1_syn3 | 93.78 | 98.04 ± 0.08 |
| | T2D-7 | 1,056 | Eggerthella lenta | 94.22 | 99.63 ± 0.03 |
| | T2D-137 | 425 | Escherichia coli | 70.35 | 99.01 ± 0.08 |
| control | Con-107 | 1,677 | Clostridiales sp. SS3/4 | 97.02 | 97.95 ± 0.06 |
| group | Con-112 | 232 | Eubacterium rectale | 90.52 | 97.56 ± 0.12 |
| enrichment | Con-129 | 1,440 | Faecalibacterium prausnitzii | 96.74 | 98.18 ± 0.04 |
| | Con-166 | 273 | Haemophilus parainfluenzae | 95.24 | 94.81 ± 0.17 |
| | Con-121 | 3,507 | Roseburia intestinalis | 92.19 | 98.90 ± 0.03 |
| | Con-113 | 345 | Roseburia inulinivorans | 94.20 | 98.21 ± 0.11 |

^{e}percentage of MLG genes in the closest species
^{f}average similarity of the closest species.

### Example 6

### Odds Ratios of Species Markers

In order to further verify the found species markers, the odds ratio of each species marker was calculated in the 344 samples above (shown in Table 8). The results showed that the species have high strength association (Odds ratio is greater than 1. Greater odds ratio is, more obviously enriched in the corresponding group of samples the species marker is).

TABLE 8
odds ratios of species markers

| | | Taxonomy assignment | Odds |
|---|---|---|---|
| Enrichment | MLG ID | (level) | ratios (95% CI) |
| T2D group | T2D-154 | Akkermansia muciniphila | 1.52 (1.05, 2.19) |
| enrichment | T2D-140 | Bacteroides intestinalis | 1.50 (1.15, 1.97) |
| | T2D-139 | Bacteroides sp. 20_3 | 1.66 (1.26, 2.20) |
| | T2D-11 | Clostridium bolteae | 5.89 (1.39, 25.0) |
| | T2D-5 | Clostridium hathewayi | 23.1 (2.08, 256.6) |
| | T2D-80 | Clostridium ramosum | 1.68 (0.97, 2.89) |
| | T2D-57 | Clostridium sp. HGF2 | 2.62 (1.14, 6.03) |
| | T2D-15 | Clostridium symbiosum | 1.13 (0.88, 1.44) |
| | T2D-1 | Desulfovibrio3 | 1.41 (0.93, 2.13) |
| | | sp. 3_1_syn | |
| | T2D-7 | Eggerthella lenta | 1.57 (0.95, 2.58) |
| | T2D-137 | Escherichia coli | 1.72 (1.16, 2.57) |
| control | Con-107 | Clostridiales sp. SS3/4 | 1.44 (1.13, 1.84) |
| group | Con-112 | Eubacterium rectale | 1.51 (1.13, 2.03) |
| enrichment | Con-129 | Faecalibacterium | 1.55 (1.19, 2.00) |
| | | prausnitzii | |
| | Con-166 | Haemophilus | 1.25 (0.93, 1.69) |
| | | parainfluenzae | |
| | Con-121 | Roseburia intestinalis | 3.10 (1.92, 5.03) |
| | Con-113 | Roseburia inulinivorans | 1.45 (1.11, 1.89) |

### Example 7

### Validation in Animal Experiment

### Method:

To measure the effects of one strain to normal mice which fed different diet by oral administration, twenty four male C57BL/6J mice (4 weeks old, Laboratorial animal Centre, Sun Yat-Sen University, China) were housed in groups of 4 per cage in a controlled environment: 12-hour daylight cycle and temperature-controlled room (22° C.) with free access to food and water. After two weeks of acclimatization, the mice were divided into 3 groups (n=8/group): a control group (group C), fed with a control chow diet (Laboratorial animal Centre, Sun Yat-Sen University, China), two groups fed a HF diet (D12492, Research Diets) which one group received bacteria (the Bacteria group, group B) and one did not (group A) during 8 weeks. A 0.2 ml dose of bacteria (10⁸ colony-forming units/0.2 ml) was administered via a stomach tube to the group B mice for 8 weeks. The energy content of the HF diet consisted of fat for 60%, carbohydrate for 20% and protein for 20%.

To measure the effects of one strain to diabetic model mice, a total of 24 male C57BL/6J mice (4 weeks old, Laboratorial animal Centre, Sun Yat-Sen University, China) were maintained in a temperature-controlled room (22° C.) on a 12-h light-dark cycle with free access to food and water. After two weeks of acclimatization, the mice were transferred to feeding a high-fat diet (D12492, Research Diets) for 8 weeks. And on the 4 weeks, they were additionally given 60 mg/kg alloxan by peritoneal injection on two consecutive days. And after the next follow 4 weeks, the mice, whose fasting serum glucose was larger than 10.0 mmol/L, were collected from them and randomly divided into two groups of 8-10 animals each. One group received bacteria (the Bacteria group, group DB) and one did not (Group Diabetes Control). A 0.2 ml dose of bacteria (10⁶˜10⁸ colony-forming units/0.2 ml) was administered via a stomach tube to the group DB of mice for 8 weeks. The mice in the Group Diabetes Control were administered 0.2 ml physiological saline solution via a stomach tube, under the same dietary and living conditions.

Body Weight was Measured Once a Week.

For each species, the inventors chosen two available strains (shown in Table 9) as examples, including type strain which has great importance for classification at the species level, and non-type strain. If the species has only one strain in taxonomy, then the inventor just chosen that one.

TABLE 9
Strains

| | | Biological Properties | | | | | |
|---|---|---|---|---|---|---|---|
| | Available | | Gram | | Oxygen | | Temperature |
| Strains | sources | Cell Shape | Staining | Motility | requirement | Habitat | Range |
| Roseburia | DSMZ, | Rod-shaped | Gram+ | Motile | Anaerobe | Host | Mesophile |
| intestinalis | | | | | | | |
| DSM 14610^{T} | | | | | | | |
| Roseburia | The | Rod-shaped | Gram+ | Motile | Anaerobe | Host | Mesophile |
| intestinalis | Wellcome | | | | | | |
| M50/1 | Trust | | | | | | |
| | Sanger | | | | | | |
| | Institute | | | | | | |
| Roseburia | DSMZ | Rod-shaped | Gram+ | Motile | Anaerobe | Host | Mesophile |
| inulinivorans | | | | | | | |
| DSM 16841^{T} | | | | | | | |
| Roseburia | The | Rod-shaped | Gram+ | Motile | Anaerobe | Host | Mesophile |
| inulinivorans | Genome | | | | | | |
| L1-83 | Institute at | | | | | | |
| | Washington | | | | | | |
| | University | | | | | | |
| Eubacterium | ATCC, | Rod-shaped | Gram+ | Motile | Anaerobe | Host | Mesophile |
| rectale ATCC | American | | | | | | |
| 33656^{T} | Type | | | | | | |
| | Culture | | | | | | |
| | Collection | | | | | | |
| Eubacterium | DSMZ | Rod-shaped | Gram+ | Motile | Anaerobe | Host | Mesophile |
| rectale DSM | | | | | | | |
| 17629 | | | | | | | |
| Haemophilus | ATCC | Rod-shaped | Gram− | Nonmotile | Facultative | Host | Mesophile |
| parainfluenzae | | | | | | | |
| ATCC | | | | | | | |
| 33392^{T} | | | | | | | |
| Haemophilus | ATCC | Rod-shaped | Gram− | Nonmotile | Facultative | Host | Mesophile |
| parainfluenzae | | | | | | | |
| ATCC | | | | | | | |
| 33966 | | | | | | | |
| Faecalibacterium | National | Rod-shaped | Gram− | Nonmotile | Anaerobe | Host | Mesophile |
| prausnitzii | Collection of | | | | | | |
| NCIMB | Industrial | | | | | | |
| 13872^{T} | Bacteria | | | | | | |
| Faecalibacterium | DSMZ | Rod-shaped | Gram− | Nonmotile | Anaerobe | Host | Mesophile |
| prausnitzii | | | | | | | |
| DSM 17677 | | | | | | | |
| Clostridiales | The | Coccus-shaped | Gram+ | Nonmotile | Anaerobe | Host | Mesophile |
| sp. SS3/4 | Wellcome | | | | | | |
| | Trust | | | | | | |
| | Sanger | | | | | | |
| | Institute | | | | | | |
| Akkermansia | DSMZ | Oval-shaped | Gram− | Nonmotile | Anaerobe | Host | Mesophile |
| muciniphila | | | | | | | |
| DSM 22959^{T} | | | | | | | |
| Bacteroides | DSMZ | Rod-shaped | Gram− | Nonmotile | Anaerobe | Host | Mesophile |
| intestinalis | | | | | | | |
| DSM 17393^{T} | | | | | | | |
| Bacteroides | J. Craig | Rod-shaped | Gram− | Nonmotile | Anaerobe | Host | Mesophile |
| intestinalis | Venter | | | | | | |
| EK2 | Institute | | | | | | |
| Clostridium | DSMZ | Rod-shaped | Gram+ | Motile | Anaerobe | Host | Mesophile |
| bolteae DSM | | | | | | | |
| 15670^{T} | | | | | | | |
| Clostridium | BEI | Rod-shaped | Gram+ | Motile | Obligate | Host | Mesophile |
| bolteae | Resources, | | | | anaerobe | | |
| WAL-14578 | Number | | | | | | |
| | HM-318 | | | | | | |
| Clostridium | DSMZ | Rod-shaped | Gram+ | Motile | Anaerobe | Host | Mesophile |
| hathewayi | | | | | | | |
| DSM | | | | | | | |
| 13479^{T} | | | | | | | |
| Clostridium | BEI | Rod-shaped | Gram+ | Motile | Obligate | Host | Mesophile |
| hathewayi | Resources, | | | | anaerobe | | |
| WAL-18680 | Number | | | | | | |
| | HM-308 | | | | | | |
| Escherichia | DSMZ | Rod-shaped | Gram− | Motile | Facultative | Host | Mesophile |
| coli DSM | | | | | | | |
| 30083^{T} | | | | | | | |
| Escherichia | ATCC | Rod-shaped | Gram− | Motile | Facultative | Host | Mesophile |
| coli ATCC | | | | | | | |
| 8739 | | | | | | | |
| Clostridium | DSMZ | Rod-shaped | Gram+ | Motile | Anaerobe | Host | Mesophile |
| ramosum | | | | | | | |
| DSM 1402^{T} | | | | | | | |
| Clostridium | ATCC | Rod-shaped | Gram+ | Motile | Anaerobe | Host | Mesophile |
| ramosum | | | | | | | |
| ATCC 25554 | | | | | | | |
| Clostridium | DSMZ | Rod-shaped | Gram+ | Motile | Anaerobe | Host | Mesophile |
| symbiosum | | | | | | | |
| DSM 934^{T} | | | | | | | |
| Clostridium | BEI HM-309 | Rod-shaped | Gram+ | Motile | Obligate | Host | Mesophile |
| symbiosum | | | | | anaerobe | | |
| WAL-14163 | | | | | | | |
| Eggerthella | DSMZ | Rod-shaped | Gram+ | Nonmotile | Anaerobe | Host | Mesophile |
| lenta DSM | | | | | | | |
| 2243^{T} | | | | | | | |
| Eggerthella | BEI | Rod-shaped | Gram+ | Nonmotile | Anaerobe | Host | Mesophile |
| lenta | Resources, | | | | | | |
| 1_ 1 _60AFAA | Number | | | | | | |
| | HM-301 | | | | | | |
| Bacteroides | BEI | Rod-shaped | Gram− | Motile | Anaerobe | Host | Mesophile |
| sp. 20_3 | Resources, | | | | | | |
| | Number | | | | | | |
| | HM-166 | | | | | | |
| Clostridium | BEI | Rod-shaped | Gram+ | Motile | Anaerobe | Host | Mesophile |
| sp. HGF2 | Resources, | | | | | | |
| | Number | | | | | | |
| | HM-287 | | | | | | |
| Desulfovibrio | Broad | Rod-shaped | Gram− | Motile | Anaerobe | Host | Mesophile |
| sp. 3_1_syn3 | Institute | | | | | | |

*^{T}: type strain;
DSMZ: Leibniz-Institute DSMZ—Deutsche Sammlung von Mikroorganismen und Zellkulturen GmbH

### Blood Parameters

Blood samples were taken at indicated time points from the retrobulbar, intraorbital, capillary plexus after 16-h fasted and following immediate centrifugation at 4° C. Plasma was separated and stored at −20° C. until analysis. Baseline Serum glucose was determined using a glucose meter (Roche Diagnostics), plasma triglycerides was measured using kits coupling enzymatic reaction and spectrophotometric detection of reaction end products, plasma insulin and glycated hemoglobin HbAlc concentrations were determined using ELISA kit (Nanjing Jiancheng Bioengineering Institute).

### Statistical Analyses

Results are presented as mean±SEM. Statistical analysis was performed by ANOVA followed by post hoc Tuckey's multiple comparison test (GraphPad Software, San Diego, Calif., USA); p<0.05 was considered as statistically significant. Correlations between parameters were assessed by Pearson's correlation test; correlations were considered significant as follows: *p<0.05,**p<0.01, ***p<0.001.

### Results

In the experimental high-fat diet was introduced at 6 weeks of age in 2/3 of the animals (n=16), and the 1/3 was maintained on the normal, low-fat diet (n=8). While half of mice fed high fat diet were treated with bacterial strains in their natural cultures by oral administration. At this stage, body weight, fasting serum glucose, serum triglyceride, serum insulin and HbAlc didn't show significant differences in all groups. Based on the following comprehensive data of body weight, fasting serum glucose, serum triglyceride, serum insulin and HbAlc, the results indicated that all of the bacteria in group B1-B6 had benefits for prevention and treatment of T2D, and all of the bacteria in group B7-B17 could accelerate T2D occurrence.

### Body Weight

As obesity is a major risk factor for insulin resistance (Seamus Crowe, et al. Pigment Epithelium-Derived Factor Contributes to Insulin Resistance in Obesity. Cell Metabolism, Volume 10, Issue 1, 40-47, doi:10.1016/j.cmet.2009.06.001, incorporated herein by reference),which induces T2D, controlling obesity occurrence have benefits for prevention of T2D.

In the growth curves, during the 8 weeks after introduction of high-fat diet, body weight increased significantly more in the high-fat diet-fed mice, which 11.5±1.4 g than in the normal diet-fed mice (4.5±0.1 g; P<0.001).And the body weight of HF fed with II strains of bacteria (group B1-B6) was significantly lower than HF group (P<0.05), which indicated that all of these strains could control obesity occurrence effectively and have benefits for prevention of T2D (FIG. A1-A6)

While mice treated with B7-B17 demonstrated increases in body weight (group B7-B17) comparing with high-fat diet-fed mice (group A) during the 8 weeks, which shown in FIG. A7-A17, and most of the increases were significant. The results shown that all of these strains could accelerate obesity occurrence and then induce T2D.

### Baseline Serum Glucose

Before the first study on normal mice (at 5 weeks of age), basal glucose was 4.30±0.59 mmol/I no difference in all groups. After 8 weeks, in the level of glucose (by 4.20±1.07 mmol/I), no difference was observed on the mice maintained normal diet. While to the group taken high-fat diet, the concentration of serum glucose increased by 8.40±0.75 mmol/l (P<0.01). And the baseline glucose level of the Group B1-B6 were lower than Group A fed HF diet only, although still higher than Group C fed normal diet. But to Group B7-B17, the case was almost reversed. This tendency continued to be progressed to the 8^{th} week (Table 10).

TABLE 10
Effects of strains administration on serum glucose in normal mice fed high-fat diet

| | | | Serum glucose (mmol/l) | | |
|---|---|---|---|---|---|
| | | | Period | | |
| Group ID | | | 0 week | 4 weeks | 8 weeks |
| Group C | | | 4.17 ± 0.85 | 4.37 ± 0.72 | 4.20 ± 1.07 |
| Group A | | | 4.36 ± 1.09 | 7.20 ± 1.11 | 8.40 ± 0.75 |
| Beneficial | Group B1 | Clostridiales sp. SS3/4 | 4.16 ± 0.32 | 5.80 ± 1.48* | 6.84 ± 1.43* |
| Markers | Group B2-1 | Eubacterium rectale ATCC | 4.58 ± 0.53 | 6.01 ± 0.73* | 6.73 ± 1.42* |
| | | 33656^{T} | | | |
| | Group B2-2 | Eubacterium rectale DSM | 4.34 ± 0.47 | 5.72 ± 1.64* | 6.68 ± 0.89* |
| | | 17629 | | | |
| | Group B3-1 | Roseburia inulinivorans | 4.33 ± 0.54 | 5.52 ± 1.79* | 6.20 ± 1.18*** |
| | | DSM 16841^{T} | | | |
| | Group B3-2 | Roseburia inulinivorans | 4.26 ± 0.44 | 5.63 ± 1.58* | 6.43 ± 0.94*** |
| | | L1-83 | | | |
| | Group B4-1 | Roseburia intestinalis DSM | 4.26 ± 0.95 | 5.87 ± 1.39* | 6.78 ± 1.20* |
| | | 14610^{T} | | | |
| | Group B4-2 | Roseburia intestinalis | 4.32 ± 0.56 | 5.65 ± 1.44* | 6.52 ± 0.91*** |
| | | M50/1 | | | |
| | Group B5-1 | Faecalibacterium | 4.27 ± 0.70 | 5.61 ± 1.51* | 6.11 ± 1.25*** |
| | | prausnitzii NCIMB 13872^{T} | | | |
| | Group B5-2 | Faecalibacterium | 4.31 ± 0.60 | 5.82 ± 1.66* | 6.24 ± 0.87*** |
| | | prausnitzii DSM 17677 | | | |
| | Group B6-1 | Haemophilus | 4.58 ± 0.58 | 5.90 ± 1.15* | 5.90 ± 0.69*** |
| | | parainfluenzae ATCC | | | |
| | | 33392^{T} | | | |
| | Group B6-2 | Haemophilus | 4.34 ± 0.49 | 5.77 ± 1.87* | 6.95 ± 0.46*** |
| | | parainfluenzae ATCC | | | |
| | | 33966 | | | |
| Harmful | Group B7-1 | Clostridium bolteae DSM | 4.10 ± 0.78 | 8.51 ± 1.85 | 9.87 ± 1.28* |
| Markers | | 15670^{T} | | | |
| | Group B7-2 | Clostridium bolteae | 4.14 ± 0.67 | 8.60 ± 1.37* | 9.94 ± 0.85* |
| | | WAL-14578 | | | |
| | Group B8-1 | Escherichia coli DSM | 4.20 ± 0.30 | 8.80 ± 1.10* | 10.90 ± 1.94** |
| | | 30083^{T} | | | |
| | Group B8-2 | Escherichia coli ATCC | 4.36 ± 0.26 | 8.94 ± 1.05* | 10.97 ± 1.68** |
| | | 8739 | | | |
| | Group B9 | Bacteroides sp. 20_3 | 4.14 ± 0.45 | 8.71 ± 1.00* | 9.83 ± 1.03* |
| | Group B10-1 | Bacteroides intestinalis | 4.50 ± 0.62 | 8.92 ± 0.74* | 10.57 ± 1.39** |
| | | DSM 17393^{T} | | | |
| | Group B10-2 | Bacteroides intestinalis | 4.41 ± 0.59 | 8.99 ± 1.51* | 10.69 ± 0.97** |
| | | EK2 | | | |
| | Group B11 | Akkermansia muciniphila | 4.51 ± 0.74 | 8.84 ± 1.35 | 9.85 ± 0.69* |
| | | DSM 22959^{T} | | | |
| | Group B12-1 | Clostridium symbiosum | 4.60 ± 0.69 | 9.20 ± 1.94* | 10.24 ± 0.66** |
| | | DSM 934^{T} | | | |
| | Group B12-2 | Clostridium symbiosum | 4.35 ± 0.50 | 9.34 ± 1.58** | 10.49 ± 0.73** |
| | | WAL-14163 | | | |
| | Group B13 | Desulfovibrio sp. 3_1_syn3 | 4.22 ± 0.47 | 8.99 ± 1.33* | 9.20 ± 0.74* |
| | Group B14 | Clostridium sp. HGF2 | 4.10 ± 0.44 | 9.97 ± 0.84** | 10.00 ± 1.22** |
| | Group B15-1 | Clostridium hathewayi | 4.02 ± 0.22 | 8.83 ± 0.72* | 9.61 ± 0.85* |
| | | DSM 13479^{T} | | | |
| | Group B15-2 | Clostridium hathewayi | 4.16 ± 0.31 | 8.61 ± 0.88** | 9.41 ± 0.76** |
| | | WAL-18680 | | | |
| | Group B16-1 | Eggerthella lenta DSM | 4.44 ± 0.20 | 8.18 ± 0.70* | 9.70 ± 0.48* |
| | | 2243^{T} | | | |
| | Group B16-2 | Eggerthella lenta | 4.51 ± 0.40 | 8.25 ± 0.64** | 9.59 ± 0.65** |
| | | 1_1_60AFAA | | | |
| | Group B17-1 | Clostridium ramosum DSM | 4.10 ± 0.54 | 9.13 ± 1.85* | 9.94 ± 0.94* |
| | | 1402^{T} | | | |
| | Group B17-2 | Clostridium ramosum | 4.20 ± 0.46 | 9.22 ± 1.74* | 9.16 ± 0.77* |
| | | ATCC 25554 | | | |

Before the later study on diabetic model mice (at 14 weeks of age), there was no difference in basal glucose in all groups. After 4 weeks, in the level of glucose on the Control Group maintained HF diet was 12.96±1.10. And the baseline glucose levels of the Group DB1-DB6 were lower than Control Group. After 8 weeks, the serum glucose of Group DB1-DB6 with 11 strains of bacteria (group B1-B6) was significantly lower than Control (P<0.05) (Table 11).

TABLE 11
Effects of strains administration on serum glucose in model mice fed high-fat diet

| | | | Serum glucose (mmol/l) | | |
|---|---|---|---|---|---|
| | | | Period | | |
| Group ID | | | 0 week | 4 weeks | 8 weeks |
| Diabetes Control | | | 11.78 ± 1.40 | 12.96 ± 1.10 | 13.48 ± 1.23 |
| Beneficial | Group DB1 | Clostridiales sp. SS3/4 | 11.34 ± 0.32 | 11.30 ± 1.48* | 11.90 ± 1.53** |
| Markers | Group DB2-1 | Eubacterium rectale | 11.98 ± 0.53 | 10.91 ± 1.33** | 11.30 ± 0.42*** |
| | | ATCC 33656^{T} | | | |
| | Group DB2-2 | Eubacterium rectale DSM | 11.89 ± 0.45 | 10.76 ± 1.58** | 11.44 ± 0.57*** |
| | | 17629 | | | |
| | Group DB3-1 | Roseburia inulinivorans | 11.81 ± 0.54 | 11.22 ± 0.79** | 11.81 ± 1.18** |
| | | DSM 16841^{T} | | | |
| | Group DB3-2 | Roseburia inulinivorans | 11.65 ± 0.56 | 11.35 ± 0.67** | 11.89 ± 1.27** |
| | | L1-83 | | | |
| | Group DB4-1 | Roseburia intestinalis | 11.11 ± 0.95 | 11.27 ± 0.79* | 11.54 ± 1.20* |
| | | DSM 14610^{T} | | | |
| | Group DB4-2 | Roseburia intestinalis | 11.34 ± 0.76 | 11.55 ± 0.66* | 11.61 ± 0.88* |
| | | M50/1 | | | |
| | Group DB5-1 | Faecalibacterium | 12.04 ± 0.70 | 11.71 ± 0.51* | 11.25 ± 1.25* |
| | | prausnitzii NCIMB 13872^{T} | | | |
| | Group DB5-2 | Faecalibacterium | 11.88 ± 0.69 | 11.87 ± 0.78* | 11.55 ± 0.75* |
| | | prausnitzii DSM 17677 | | | |
| | Group DB6-1 | Haemophilus | 12.36 ± 0.58 | 10.90 ± 1.15* | 12.28 ± 1.69* |
| | | parainfluenzae ATCC | | | |
| | | 33392^{T} | | | |
| | Group DB6-2 | Haemophilus | 12.17 ± 0.71 | 11.27 ± 1.24* | 12.41 ± 1.52* |
| | | parainfluenzae ATCC | | | |
| | | 33966 | | | |
| Harmful | Group DB7-1 | Clostridium bolteae DSM | 11.95 ± 1.18 | 13.82 ± 1.05* | 14.68 ± 0.94* |
| Markers | | 15670^{T} | | | |
| | Group DB7-2 | Clostridium bolteae | 12.14 ± 1.16 | 13.67 ± 0.83* | 14.54 ± 0.85* |
| | | WAL-14578 | | | |
| | Group DB8-1 | Escherichia coli DSM | 12.15 ± 1.10 | 14.58 ± 1.10** | 15.89 ± 1.28** |
| | | 30083^{T} | | | |
| | Group DB8-2 | Escherichia coli ATCC | 11.91 ± 0.84 | 14.79 ± 0.86** | 15.99 ± 1.05** |
| | | 8739 | | | |
| | Group DB9 | Bacteroides sp. 20_3 | 11.65 ± 1.15 | 13.88 ± 1.50* | 14.56 ± 2.03* |
| | Group DB10-1 | Bacteroides intestinalis | 11.74 ± 0.62 | 14.52 ± 1.74* | 15.10 ± 1.39* |
| | | DSM 17393^{T} | | | |
| | Group DB10-2 | Bacteroides intestinalis | 11.88 ± 0.35 | 13.97 ± 0.61* | 15.46 ± 1.24* |
| | | EK2 | | | |
| | Group DB11 | Akkermansia muciniphila | 11.68 ± 0.74 | 13.91 ± 0.55* | 14.92 ± 0.69* |
| | | DSM 22959^{T} | | | |
| | Group DB12-1 | Clostridium symbiosum | 12.26 ± 0.69 | 13.79 ± 0.95 | 14.88 ± 0.66** |
| | | DSM 934^{T} | | | |
| | Group DB12-2 | Clostridium symbiosum | 11.96 ± 0.55 | 13.68 ± 0.87 | 14.59 ± 0.87* |
| | | WAL-14163 | | | |
| | Group DB13 | Desulfovibrio sp. | 11.72 ± 0.87 | 13.66 ± 0.33 | 14.47 ± 0.33* |
| | | 3_1_syn3 | | | |
| | Group DB14 | Clostridium sp. HGF2 | 12.58 ± 0.44 | 14.61 ± 0.72** | 15.08 ± 0.82** |
| | Group DB15-1 | Clostridium hathewayi | 11.71 ± 0.92 | 13.99 ± 0.84* | 14.71 ± 0.74* |
| | | DSM 13479^{T} | | | |
| | Group DB15-2 | Clostridium hathewayi | 11.99 ± 0.63 | 13.86 ± 0.75* | 14.63 ± 0.91* |
| | | WAL-18680 | | | |
| | Group DB16-1 | Eggerthella lenta DSM | 11.94 ± 1.20 | 13.72 ± 0.44 | 14.89 ± 1.48* |
| | | 2243^{T} | | | |
| | Group DB16-2 | Eggerthella lenta | 11.97 ± 0.96 | 13.83 ± 0.56 | 14.98 ± 1.33* |
| | | 1_1_60AFAA | | | |
| | Group DB17-1 | Clostridium ramosum | 11.82 ± 0.54 | 14.00 ± 0.85* | 15.05 ± 0.94** |
| | | DSM 1402^{T} | | | |
| | Group DB17-2 | Clostridium ramosum | 11.73 ± 0.46 | 14.19 ± 0.68* | 15.26 ± 1.21** |
| | | ATCC 25554 | | | |

### Baseline Serum Triglycerides, Insulin and HbAlc

At 5 weeks of age, triglycerides, insulin and HbAlc were not different among all groups. After 8 weeks, no difference was observed on maintained normal diet. While Serum triglycerides (by 1.31±0.35 mmol/L), insulin (by 14.31+2.01 mlU·L⁻¹) level and HbAlc (by 5.41±0.17%) were all significantly increased (P<0.01) in the Group A (HF diet), and they were decreased by B1-B6 administration compared to the HF diet. But the inventors were unable to observe similar decrease on Group DB7-DB17 (Table 12).

TABLE 12
Effects of strains administration on triglycerides, insulin and HbA1c in normal mice fed
high-fat diet

| | | | Triglycerides | Insulin | |
|---|---|---|---|---|---|
| Group ID | | | (mmol/L) | (mIU · L−1) | HbA1c (%) |
| Group C | | | 0.70 ± 0.32 | 8.27 ± 1.50 | 4.26 ± 0.29 |
| Group A | | | 1.31 ± 0.35 | 14.31 ± 2.01 | 5.41 ± 0.17 |
| Beneficial | Group B1 | Clostridiales sp. SS3/4 | 0.75 ± 0.26** | 12.38 ± 1.89* | 5.12 ± 0.21** |
| Markers | Group B2-1 | Eubacterium rectale | 0.90 ± 0.14** | 10.89 ± 2.56** | 4.91 ± 0.14*** |
| | | ATCC 33656^{T} | | | |
| | Group B2-2 | Eubacterium rectale | 0.86 ± 0.21** | 10.80 ± 1.37** | 4.82 ± 0.09*** |
| | | DSM 17629 | | | |
| | Group B3-1 | Roseburia | 0.83 ± 0.05** | 10.54 ± 3.38* | 5.18 ± 0.16** |
| | | inulinivorans DSM | | | |
| | | 16841^{T} | | | |
| | Group B3-2 | Roseburia | 0.74 ± 0.09** | 10.49 ± 3.24* | 5.12 ± 0.13** |
| | | inulinivorans L1-83 | | | |
| | Group B4-1 | Roseburia intestinalis | 0.75 ± 0.11** | 12.33 ± 1.42* | 5.09 ± 0.30* |
| | | DSM 14610^{T} | | | |
| | Group B4-2 | Roseburia intestinalis | 0.73 ± 0.08** | 12.54 ± 1.18* | 5.11 ± 0.27* |
| | | M50/1 | | | |
| | Group B5-1 | Faecalibacterium | 0.96 ± 0.27* | 11.11 ± 3.04* | 5.11 ± 0.34* |
| | | prausnitzii NCIMB | | | |
| | | 13872^{T} | | | |
| | Group B5-2 | Faecalibacterium | 0.99 ± 0.31* | 11.00 ± 2.98* | 5.14 ± 0.29* |
| | | prausnitzii DSM 17677 | | | |
| | Group B6-1 | Haemophilus | 0.94 ± 0.24* | 11.67 ± 2.66* | 5.03 ± 0.31* |
| | | parainfluenzae ATCC | | | |
| | | 33392^{T} | | | |
| | Group B6-2 | Haemophilus | 0.96 ± 0.29* | 11.75 ± 2.53* | 5.10 ± 0.23** |
| | | parainfluenzae ATCC | | | |
| | | 33966 | | | |
| Harmful | Group B7-1 | Clostridium bolteae | 1.63 ± 0.10* | 16.92 ± 1.88* | 6.08 ± 0.74* |
| Markers | | DSM 15670^{T} | | | |
| | Group B7-2 | Clostridium bolteae | 1.61 ± 0.14* | 16.78 ± 1.67* | 6.17 ± 0.83* |
| | | WAL-14578 | | | |
| | Group B8-1 | Escherichia coli | 1.52 ± 0.07* | 17.77 ± 2.50** | 5.90 ± 0.49* |
| | | DSM 30083^{T} | | | |
| | Group B8-2 | Escherichia coli | 1.51 ± 0.11* | 17.81 ± 1.99** | 5.97 ± 0.44* |
| | | ATCC 8739 | | | |
| | Group B9 | Bacteroides sp. | 1.72 ± 0.14** | 16.54 ± 1.27* | 5.67 ± 0.27* |
| | | 20_3 | | | |
| | Group B10-1 | Bacteroides | 1.73 ± 0.38* | 15.92 ± 0.42* | 5.93 ± 0.44* |
| | | intestinalis DSM | | | |
| | | 17393^{T} | | | |
| | Group B10-2 | Bacteroides | 1.65 ± 0.50* | 16.63 ± 0.64* | 5.90 ± 0.31* |
| | | intestinalis EK2 | | | |
| | Group B11 | Akkermansia | 1.66 ± 0.31* | 16.03 ± 1.39* | 5.65 ± 0.22* |
| | | muciniphila DSM | | | |
| | | 22959^{T} | | | |
| | Group B12-1 | Clostridium | 1.61 ± 0.15* | 16.11 ± 0.79* | 5.77 ± 0.42* |
| | | symbiosum DSM | | | |
| | | 934^{T} | | | |
| | Group B12-2 | Clostridium | 1.57 ± 0.33* | 16.24 ± 0.93* | 5.79 ± 0.36* |
| | | symbiosum | | | |
| | | WAL-14163 | | | |
| | Group B13 | Desulfovibrio sp. | 1.56 ± 0.05* | 16.59 ± 0.72* | 5.80 ± 0.40* |
| | | 3_1_syn3 | | | |
| | Group B14 | Clostridium sp. | 1.62 ± 0.27* | 17.33 ± 2.43* | 6.06 ± 0.49** |
| | | HGF2 | | | |
| | Group B15-1 | Clostridium | 1.77 ± 0.23* | 16.16 ± 1.20* | 6.12 ± 0.88* |
| | | hathewayi DSM | | | |
| | | 13479^{T} | | | |
| | Group B15-2 | Clostridium | 1.69 ± 0.41* | 16.43 ± 1.02* | 6.25 ± 0.79* |
| | | hathewayi | | | |
| | | WAL-18680 | | | |
| | Group B16-1 | Eggerthella lenta | 1.60 ± 0.18* | 16.33 ± 2.00* | 5.71 ± 0.34* |
| | | DSM 2243^{T} | | | |
| | Group B16-2 | Eggerthella lenta | 1.65 ± 0.26* | 16.51 ± 1.90* | 5.79 ± 0.32* |
| | | 1_1_60AFAA | | | |
| | Group B17-1 | Clostridium | 1.67 ± 0.33* | 17.13 ± 1.66** | 5.95 ± 0.52* |
| | | ramosum DSM | | | |
| | | 1402^{T} | | | |
| | Group B17-2 | Clostridium | 1.69 ± 0.21* | 17.26 ± 1.21** | 6.08 ± 0.69* |
| | | ramosum ATCC | | | |
| | | 25554 | | | |

The effect of 17 Bacteria strains on triglycerides, insulin and HbAlc in model mice were measured. All the B I to B6-treated groups had significantly lower serum triglycerides, insulin and HbAlc concentrations than those of the control group. But the inventors were unable to observe similar decrease on Group DB7-DB17 (Table 13).

TABLE 13
Effects of strains administration on triglycerides, insulin and HbA1c in model mice fed
high-fat diet

| | | | Triglycerides | Insulin | HbA1c |
|---|---|---|---|---|---|
| Group ID | | | (mmol/L) | (mIU · L−1) | (%) |
| Diabetes Control | | | 1.50 ± 0.15 | 20.31 ± 1.70 | 6.88 ± 1.19 |
| Beneficial | Group DB1 | Clostridiales sp. SS3/4 | 1.12 ± 0.23** | 18.38 ± 1.92* | 5.04 ± 1.87* |
| Markers | Group DB2-1 | Eubacterium rectale ATCC | 1.29 ± 0.24* | 16.66 ± 2.19** | 5.13 ± 1.44* |
| | | 33656^{T} | | | |
| | Group DB2-2 | Eubacterium rectale DSM | 1.24 ± 0.30* | 16.54 ± 1.44** | 5.17 ± 1.25* |
| | | 17629 | | | |
| | Group DB3-1 | Roseburia inulinivorans | 1.26 ± 0.13** | 17.00 ± 3.02* | 5.77 ± 0.92* |
| | | DSM 16841^{T} | | | |
| | Group DB3-2 | Roseburia inulinivorans | 1.22 ± 0.09** | 17.05 ± 2.66* | 5.69 ± 0.97* |
| | | L1-83 | | | |
| | Group DB4-1 | Roseburia intestinalis DSM | 1.28 ± 0.12* | 18.17 ± 2.15* | 5.32 ± 1.20* |
| | | 14610^{T} | | | |
| | Group DB4-2 | Roseburia intestinalis | 1.38 ± 0.29* | 18.54 ± 1.37* | 5.04 ± 1.90* |
| | | M50/1 | | | |
| | Group DB5-1 | Faecalibacterium | 1.33 ± 0.18* | 18.86 ± 2.67* | 6.01 ± 0.42* |
| | | prausnitzii NCIMB 13872^{T} | | | |
| | Group DB5-2 | Faecalibacterium | 1.31 ± 0.15* | 18.61 ± 1.97* | 6.03 ± 0.21* |
| | | prausnitzii DSM 17677 | | | |
| | Group DB6-1 | Haemophilus | 1.37 ± 0.10* | 18.92 ± 0.88* | 5.94 ± 0.61* |
| | | parainfluenzae ATCC | | | |
| | | 33392^{T} | | | |
| | Group DB6-2 | Haemophilus | 1.35 ± 0.08* | 18.61 ± 1.96* | 6.02 ± 0.45* |
| | | parainfluenzae ATCC | | | |
| | | 33966 | | | |
| Harmful | Group DB7-1 | Clostridium bolteae DSM | 1.68 ± 0.04* | 21.97 ± 3.20* | 7.98 ± 1.00* |
| Markers | | 15670^{T} | | | |
| | Group DB7-2 | Clostridium bolteae | 1.65 ± 0.07* | 21.89 ± 2.26* | 8.11 ± 1.31* |
| | | WAL-14578 | | | |
| | Group DB8-1 | Escherichia coli DSM | 1.95 ± 0.27** | 24.55 ± 3.12** | 8.51 ± 1.70* |
| | | 30083^{T} | | | |
| | Group DB8-2 | Escherichia coli ATCC | 1.99 ± 0.21** | 24.64 ± 2.34** | 8.60 ± 1.30* |
| | | 8739 | | | |
| | Group DB9 | Bacteroides sp. 20_3 | 1.89 ± 0.14** | 21.80 ± 2.90* | 8.09 ± 1.98* |
| | Group DB10-1 | Bacteroides intestinalis | 1.78 ± 0.20** | 21.71 ± 0.90* | 7.94 ± 1.05* |
| | | DSM 17393^{T} | | | |
| | Group DB10-2 | Bacteroides intestinalis | 1.85 ± 0.15** | 21.80 ± 0.59* | 8.09 ± 1.21* |
| | | EK2 | | | |
| | Group DB11 | Akkermansia muciniphila | 1.70 ± 0.19** | 24.69 ± 2.77** | 8.45 ± 1.45* |
| | | DSM 22959^{T} | | | |
| | Group DB12-1 | Clostridium symbiosum | 1.63 ± 0.05* | 21.78 ± 1.75* | 8.21 ± 1.10* |
| | | DSM 934^{T} | | | |
| | Group DB12-2 | Clostridium symbiosum | 1.67 ± 0.09* | 21.69 ± 0.92* | 8.43 ± 1.29* |
| | | WAL-14163 | | | |
| | Group DB13 | Desulfovibrio sp. | 1.73 ± 0.25* | 21.93 ± 1.53* | 9.36 ± 1.90** |
| | 3_1_syn3 | | | | |
| | Group DB14 | Clostridium sp. HGF2 | 1.83 ± 0.11** | 22.61 ± 2.20** | 9.18 ± 1.27** |
| | Group DB15-1 | Clostridium hathewayi | 1.64 ± 0.10* | 21.75 ± 1.25* | 8.82 ± 0.90** |
| | | DSM 13479^{T} | | | |
| | Group DB15-2 | Clostridium hathewayi | 1.68 ± 0.16* | 21.68 ± 0.88* | 8.97 ± 0.51** |
| | | WAL-18680 | | | |
| | Group DB16-1 | Eggerthella lenta DSM | 1.66 ± 0.15* | 22.22 ± 1.69* | 7.96 ± 0.99* |
| | | 2243^{T} | | | |
| | Group DB16-2 | Eggerthella lenta | 1.69 ± 0.16* | 22.35 ± 1.27* | 7.84 ± 0.83* |
| | | 1_1_60AFAA | | | |
| | Group DB17-1 | Clostridium ramosum DSM | 1.88 ± 0.34* | 21.90 ± 1.21* | 8.28 ± 1.22* |
| | | 1402^{T} | | | |
| | Group DB17-2 | Clostridium ramosum | 1.81 ± 0.29* | 21.83 ± 0.97* | 8.37 ± 1.38* |
| | | ATCC 25554 | | | |

The specific embodiment of the present invention has been described in detail, and skilled in the art will understand the same. According to the published guidance, modifications and replacement of those details can be performed. These changes are within the scope of protection of the present invention. The full scope of the present invention is given by the appended claims and any of its equivalents.

In the description, the term “one embodiment”, “some embodiments”, “schematic embodiment”, “example”, “specific examples” or “some examples” means the specific features, structures, materials or characteristics are included by at least one embodiment or example in the present invention. In the description, the schematic representation of the terms above does not necessarily mean the same embodiment or example. Moreover, the description of the specific features, structure, materials, or characteristics can be combined with in any one or more embodiments or samples in a suitable way.

What is claimed is:

1. A method of using a group of microbes to determine an abnormal condition wherein the group comprising Akkermansia muciniphila, Bacteroides intestinalis, Bacteroides sp. 20_{—}3, Clostridium bolteae, Clostridium hathewayi, Clostridium ramosum, Clostridium sp. HGF2, Clostridium symbiosum, Desulfovibrio sp. 3_{—}1_syn3, Eggerthella lenta, Escherichia coli, Clostridiales sp. SS3/4, Eubacterium rectale, Faecalibacterium prausnitzii, Haemophilus parainfluenzae, Roseburia intestinalis and Roseburia inulinivorans.

2. A method to determine abnormal condition in a subject comprising the step of determining presence or absence of the group of microbes in claim 1 in a gut microbiota of the subject.

3. The method of claim 2, wherein the abnormal condition is Diabetes.

4. The method of claim 2, wherein an excreta of the subject is assayed to determine the presence or absence of the group of microbes.

5. The method of claim 2, wherein determining the presence or absence of the group of microbes in claim 1 further comprises:
isolating nucleic acid sample from the excreta of the subject;
constructing a DNA library based on the obtaining nucleic acid sample;
sequencing the DNA library to obtain a sequencing result; and
determining the presence or absence of the group of microbes, based on the sequencing result.

6. The method of claim 5, wherein the sequencing step is conducted by means of second-generation sequencing method or third-generation sequencing method.

7. The method of claim 5, wherein the sequencing step is conducted by means of at least one apparatus selected from Hiseq 2000, SOLID, 454, and True Single Molecule Sequencing.

8. The method of claim 5, wherein determining the presence or absence of the group of microbes further comprises:
aligning the sequencing result against the group of microbes; and
determining the presence or absence of the group of microbes based on the alignment result.

9. The method of claim 8, wherein the step of aligning is conducted by means of at least one of SOAP 2 and MAQ.

10. The method of claim 2, further comprising the steps of:
determining relative abundances of the group of microbes; and
comparing the abundances with predicted critical values.

11. A system to assay abnormal condition in a subject comprising:
nucleic acid sample isolation apparatus, which adapted to isolate nucleic acid sample from the subject;
sequencing apparatus, which connected to the nucleic acid sample isolation apparatus and adapted to sequence the nucleic acid sample, to obtain a sequencing result; and
alignment apparatus, which connect to the sequencing apparatus, and adapted to align the sequencing result against the group of microbes in claim 1 in such a way that determine the presence or absence of the group of microbes in claim 1 based on the alignment result.

12. The system of claim 11, wherein the abnormal condition is Diabetes.

13. The system of claim 11, wherein an excreta of the subject is assayed to determine the presence or absence of the group of microbes.

14. The system of claim 1, wherein the sequencing apparatus is adapted to carry out second-generation sequencing method or third-generation sequencing method.

15. The system of claim 14, wherein the sequencing apparatus is adapted to carry out at least one apparatus selected from Hiseq 2000, SOLID, 454, and True Single Molecule Sequencing.

16. The system of claim 11, wherein the alignment apparatus is at least one of SOAP 2 and MAQ.

17. A kit for determining abnormal condition comprising reagents adapted to determine the group of microbes in claim 1.

18. The usage of biomarkers as target for screening medicaments to treat or prevent Type 2 Diabetes, in which the biomarkers are the group of microbes in claim 1.

19. The method of claim 2, wherein the abnormal condition is Type 2 Diabetes.

20. The method of claim 2, wherein an excreta of the subject is assayed to determine the presence or absence of the group of microbes, wherein the excreta is a faecal sample.

21. The system of claim 11, wherein the abnormal condition is Type 2 Diabetes.

22. The system of claim 11, wherein an excreta of the subject is assayed to determine the presence or absence of the group of microbes wherein the excreta is a faecal sample.

23. A method of using a group of microbes to treat or prevent an abnormal condition wherein the group comprising Clostridiales sp. SS3/4, Eubacterium rectale, Faecalibacterium prausnitzii, Haemophilus parainfluenzae, Roseburia intestinalis and Roseburia inulinivorans.

24. The method of claim 23, where the abnormal condition is Diabetes.

25. The method of claim 23, where the abnormal condition is T2D.

26. The method of claim 23, where at least one member of the group of microbes are used in a food or pharmaceutical composition.

27. The method of claim 1, where any member of the group of microbes or in any combination thereof is used.

28. The method of claim 23, where any member of the group of microbes or in any combination thereof is used.
